(19) United States
(12) Patent Application Publication
(10) Pub. No.: US 2024/0361328 A1
(43) Pub. Date: Oct. 31, 2024

(54) APPLICATION OF MULTIMER IN DETECTION AND PREPARATION OF CAR-EXPRESSING CELL

(71) Applicant: JUN HUANG, CHICAGO, IL (US)

(72) Inventors: JUN HUANG, CHICAGO, IL (US); YIFEI HU, CHICAGO, IL (US)

(21) Appl. No.: 18/646,665

(22) Filed: Apr. 25, 2024

Related U.S. Application Data
(63) Continuation of application No. PCT/CN2022/127159, filed on Oct. 24, 2022.

(30) Foreign Application Priority Data
Oct. 25, 2021 (CN) 202111243790.2

(51) Int. Cl. G01N 33/58; C07K 1/13; C07K 16/28; C07K 16/30; C07K 16/32; C12N 5/0783; G01N 33/569

(52) U.S. Cl. CPC G01N 33/58; C07K 1/13; C07K 16/2803; C07K 16/30; C07K 16/32; C12N 5/0636; G01N 33/56972; G01N 33/582; C07K 2317/92; C07K 2319/22; C07K 2319/33; C07K 2319/60

(57) ABSTRACT

The present invention discloses a versatile application of a multimer technology for detecting, manufacturing, and profiling chimeric antigen receptor (CAR)-expressing cells. These antigen-multimers exhibit remarkable specificity, sensitivity, and precision in identifying CAR cells. Moreover, they facilitate over 100-fold magnetic enrichment of rare CAR-T cells, enhancing their detectability. Beyond CAR detection, the multimer technology selectively stimulates CAR-expressing cells, both in soluble and surface-bound formats, during CAR-T cell manufacturing. Unlike existing technologies (such as anti-CD28/CD3 magnetic beads) that non-specifically stimulate and expand all T cells, the multimer approach ensures that only CAR-expressing cells are targeted. Consequently, this yields a CAR-T cell product with significantly higher purity, enhancing treatment efficacy. Furthermore, the multimer technology offers flexibility in CAR detection through nucleotide labeling. This enables high-dimensional CAR-T cell profiling via single-cell multi-omics analyses. Antigen multimers can be seamlessly adapted to other CAR systems by switching the antigen ligand.

### CROSS REFERENCE OF RELATED APPLICATIONS

The present application is a continuation-application of International Patent Application (PCT) No. PCT/CN2021/127159 filed on Oct. 24, 2022, which claims foreign priority of Chinese Patent Application No. 202111243790.2, filed on Oct. 25, 2021, the entire contents of which are hereby incorporated by reference.

### SEQUENCE LISTING

The instant application contains a Sequence Listing which has been submitted electronically in ST.26 format and is hereby incorporated by reference in its entirety. Said ST.26 copy, created on 24 Apr. 2024, is named Application of Multimer.xml and is 8,487 bytes in size.

### TECHNICAL FIELD

The present invention relates to the field of biotechnology, and specifically, refers to an application of multimer in detection and preparation of CAR-expressing cells.

### BACKGROUND

Chimeric antigen receptors (CARs) are synthetic proteins designed to recognize specific antigens. CAR-T cells, which have been genetically transduced with CARs, can effectively recognize, target, and eliminate cancer cells expressing the corresponding antigen. CAR-T cells play a crucial role in precision immunotherapy, enhancing the immune system's ability to combat cancer.

T cells, also known as T lymphocytes, serve as the foundation for creating CAR-T cells. These T cells are first activated and then genetically modified with a chimeric antigen receptor (CAR). The unique properties of CAR-T cells include their ability to specifically recognize cancer cells within the body and release a substantial amount of various effector molecules through an immune response. As a result, CAR-T cells efficiently target and eliminate cancer cells, ultimately achieving the goal of curing some malignant tumors.

Given the rapid advancement of CAR-T cell therapy, there is an urgent need for a highly specific, sensitive, precise, and multifunctional CAR staining and detection reagent to fulfill the needs of CAR-T cell detection in research, industry, and clinics. This reagent is valuable for biochemical analysis, preclinical studies, patient specimen analysis, and innovative CAR development. However, existing CAR-staining reagents have limitations. Generic CAR-staining reagents (polyclonal anti-IgG antibodies and Protein L) that bind the CAR scFv suffer from non-specific binding, incompatibility with other antibodies, and multi-step staining procedures. Specific CAR-staining reagents (target antigen and anti-idiotype antibodies) may be commercially available but are often limited by protein instability and high developmental cost. Additionally, all existing CAR-staining reagents are restricted by ≤2-valency binding. Therefore, it is of great importance in developing a CAR staining reagent with higher affinity and superior detecting capability.

The present China patent discloses “One Multimeric Binding Reagent” (CN107847544A), which can potentially solve the above-mentioned problems in this field.

In addition, the current methodology employed for manufacturing CAR-T cells involves stimulating peripheral blood T cells collected from patients using magnetic bead-conjugated anti-CD3/CD28 antibodies. The process includes transducing anti-CD19 CAR with lentivirus and subsequently stimulating and expanding CAR-T cells generated through the same magnetic bead-conjugated anti-CD3/CD28 antibodies. However, this technology faces several challenges. First, there is toxicity associated with the unseparated magnetic beads for in vivo application. Second, due to the incomplete CAR transfection by lentivirus, non-CAR-T cells are nonspecifically expanded, resulting in a low yield of CAR-T cells. Additionally, the universal stimulation effect induced by anti-CD3/CD28 antibodies affects the phenotype and function of the entire T cell populations, regardless of CAR expression. Consequently, the purity and function of the current commercial CAR-T cell product are compromised, with only approximately 30% of the cell population being CAR-T cells, while the majority (around 70%) consists of non-CAR-T cells (as shown in FIG. 11B). Overall, these limitations hinder the clinical efficacy of CAR-T cell therapy, making it an urgent issue to address in CAR-T cell manufacturing.

### SUMMARY

The present invention can overcome the above-mentioned issues in the CAR field by introducing a multimer for detecting and manufacturing CAR-expressing cells. Herein, these CAR-expressing cells encompass CAR-T cells, CAR-NK cells, CAR-macrophages, and other types of CAR-expressing cells.

The present invention initially provides a CAR staining reagent, which consists of a complex involving a multimer and an antigen. The antigen is a biotin-tagged specific antigen molecule that exhibits specific binding affinity to the CAR intended for detection.

The multimer comprises:
1) a scaffold protein, which is comprised of four protein subunits; a scaffold protein is composed of functional protein subunits; or a scaffold protein is composed of a mixture of functional and dysfunctional protein subunits (i.e. the protein subunits are functional units, or the protein subunits are a mixture of functional and dysfunctional units); the functional protein subunit is derived from a streptavidin and has no biotin-binding activity; the scaffold protein has at least one functional protein subunit, which C-terminus has a cysteine and a biotin tag; the amino acid sequence of the functional protein subunit is set forth in SEQ ID NO:1 or SEQ ID NO:2; the number of the dysfunctional protein subunit is not greater than 2, and the amino acid sequence is set forth in SEQ ID NO: 3.
2) A streptavidin binds to the biotin tag of the scaffold protein. The streptavidin can bind 1 to 3 biotin-tagged antigens. The streptavidin may or may not have detectable labels.

It is noteworthy that the sequence SEQ ID NO:1 of the multimer and protein subunit of the CAR staining reagent is identical to the sequence disclosed in the China Patent “One Multimeric Binding Reagent” (CN107847544A). The present invention further demonstrates the applications of the reagent in the staining of CAR-expressing cells.

Preferably, the CAR to be detected has property of low binding affinity.

Preferably, the affinity K_{D} value of the CAR to be detected is not less than 0.1 nM. Optionally, the CAR to be detected is CD19 CAR.

Optionally, the CAR to be detected is a variant CAR which is against the identical antigen molecule, or a mixture of different CARs which are against the identical antigen molecule.

Preferably, the working temperature of the CAR staining reagent is 0˜25° C., and more preferably to be 4° C.

Secondly, the present invention provides a specific cell expansion and manufacturing reagent for CAR-expressing cells, which is comprised of the complex of an antigen and a multimer.

The antigen is the antigen expressed on a target cell surface, which can be specifically recognized by the CAR.

The multimer comprises:
1) a scaffold protein, which is comprised of four protein subunits; a scaffold protein is composed of functional protein subunits; or a scaffold protein is composed of a mixture of functional and dysfunctional protein subunits; the functional protein subunit is derived from a streptavidin and has no biotin-binding activity; the scaffold protein has at least one functional protein subunit, which C-terminus has a cysteine and a biotin tag; the amino acid sequence of the functional protein subunit is set forth in SEQ ID NO:1 or SEQ ID NO:2; the number of the dysfunctional protein subunit is not greater than 2, and the amino acid sequence is set forth in SEQ ID NO:3.
2) A streptavidin binds to the biotin tag of the scaffold protein. The streptavidin can bind 1 to 3 biotin-tagged antigens. The streptavidin may or may not have detectable labels.

It is noteworthy that the multimer includes pentamer, octamer, dodecamer, icosamer, or other forms of multimers, comprising by various numbers of scaffold subunits, streptavidins and antigens. One scaffold subunit can bind to multiple streptavidins, and a streptavidin can bind to more than one scaffold subunits. Various compositions of multimers can be produced with different combinations. The present invention not only includes the applications of dodecamers in the specific expansion of CAR-expressing cells, but also the similar applications of other compositions of multimers, each with varying efficacy.

Preferably, the reagent is used at the temperature of 30˜40° C. and preferably at 37° C.

Optionally, the multimer is dodecamer.

Another CAR-expressing cell specific expansion reagent developed in the present invention comprises a streptavidin, and the streptavidin binds to 2˜4 antigen via biotin tag. The antigen is the antigen molecule expressed on a target cell surface and that can be specially recognized by CAR. The CAR-expressing cell specific expansion reagent is thus named antigen streptavidin tetramer. The working temperature is similarly at 30˜40° C. and preferably at 37° C.

The present invention provides the application of the CAR-expressing cell specific expansion reagent in the manufacturing of CAR-T cells.

Specifically, a CAR-T cell specific expansion methodology is provided, which comprises the following steps: the CAR-expressing cell specific expansion reagent is supplemented to the cultured CAR-T cell population and render the manufactured CAR-T cells to meet the preset therapeutic standard. Generally, the reagent accompanies with CAR-T cell growth to provide continuous stimulation to the expansion of specific CAR-T cells.

Thirdly, the present invention provides a CAR detection reagent, comprising the oligo nucleotide-labeled antigen-multimer. The antigen-multimer is the complex of antigen and multimer:

The antigen is the antigen molecule expressed on a target cell surface, which can be specifically recognized by CAR.

The multimer comprises:
1) a scaffold protein, which is comprised of four protein subunits; a scaffold protein is composed of functional protein subunits; or a scaffold protein is composed of a mixture of functional and dysfunctional protein subunits; the functional protein subunit is derived from a streptavidin and has no biotin-binding activity; the scaffold protein has at least one functional protein subunit, which C-terminus has a cysteine and a biotin tag; the amino acid sequence of the functional protein subunit is set forth in SEQ ID NO:1 or SEQ ID NO:2; the number of the dysfunctional protein subunit is not greater than 2, and the amino acid sequence is set forth in SEQ ID NO:3.
2) A streptavidin binds to the biotin tag of the scaffold protein. The streptavidin can bind 1 to 3 biotin-tagged antigens. The streptavidin may or may not have detectable labels.

Another CAR-expressing cell specific expansion reagent provided in the present invention comprises an unlabeled, fluorescence-labeled, or oligo nucleotide-labeled streptavidin, and the streptavidin binds to 2˜4 antigen via biotin tag. The antigen is the antigen expressed on a target cell surface and that can be specially recognized by CAR. The CAR-expressing cell specific expansion reagent is thus named as antigen-streptavidin tetramer.

The present invention provides a CAR expression detection methodology, which comprises the following steps: The CAR is stained and detected with the CAR expression detection reagent, and the CAR expression is analyzed with single cell sequencing.

In addition, the present invention provides a site-specific biotinylating peptide, which is sited in the antigen peptide sequence and can render the antigen peptide to be site-specifically biotinylated. The sequence is set forth in SEQ ID No.7.

An CD19 antigen-multimer, which is the complex of antigen and multimer. The antigen is the CD19 molecule containing a biotin tag. The amino acid sequence is set forth in SEQ ID No.4. The CD19 molecule binds to tetramer or other forms of multimer through its biotin tag. The tetramer is unlabeled, fluorescence-labeled, or oligo nucleotide-labeled streptavidin.

The multimer comprises:
1) a scaffold protein, which is comprised of four protein subunits; a scaffold protein is composed of functional protein subunits; or a scaffold protein is composed of a mixture of functional and dysfunctional protein subunits; the functional protein subunit is derived from a streptavidin and has no biotin-binding activity; the scaffold protein has at least one functional protein subunit, which C-terminus has a cysteine and a biotin tag; the amino acid sequence of the functional protein subunit is set forth in SEQ ID NO:1 or SEQ ID NO:2; the number of the dysfunctional protein subunit is not greater than 2, and the amino acid sequence is set forth in SEQ ID NO:3.
2) A streptavidin binds to the biotin tag of the scaffold protein. The streptavidin can bind 1 to 3 biotin-tagged antigens. The streptavidin may or may not have detectable labels.

An HER2 antigen-multimer, which is the complex of antigen and multimer. The antigen is the HER2 molecule containing a biotin tag. The amino acid sequence is set forth in SEQ ID No.5. The HER2 molecule binds to tetramer or other forms of multimer through its biotin tag. The tetramer is unlabeled, fluorescence-labeled, or oligo nucleotide-labeled streptavidin.

The multimer comprises:
1) a scaffold protein, which is comprised of four protein subunits; a scaffold protein is composed of functional protein subunits; or a scaffold protein is composed of a mixture of functional and dysfunctional protein subunits; the functional protein subunit is derived from a streptavidin and has no biotin-binding activity; the scaffold protein has at least one functional protein subunit, which C-terminus has a cysteine and a biotin tag; the amino acid sequence of the functional protein subunit is set forth in SEQ ID NO:1 or SEQ ID NO:2; the number of the dysfunctional protein subunit is not greater than 2, and the amino acid sequence is set forth in SEQ ID NO:3.
2) A streptavidin binds to the biotin tag of the scaffold protein. The streptavidin can bind 1 to 3 biotin-tagged antigens. The streptavidin may or may not have detectable labels.

An Tn glycoside antigen-multimer, which is the complex of antigen and multimer. The antigen is the Tn polypeptide containing a biotin tag. The amino acid sequence is set forth in SEQ ID No.6. The Tn antigen peptide binds to tetramer or other forms of multimer through its biotin tag. The tetramer is unlabeled, fluorescence-labeled, or oligo nucleotide-labeled streptavidin.

The multimer comprises:
1) a scaffold protein, which is comprised of four protein subunits; a scaffold protein is composed of functional protein subunits; or a scaffold protein is composed of a mixture of functional and dysfunctional protein subunits; the functional protein subunit is derived from a streptavidin and has no biotin-binding activity; the scaffold protein has at least one functional protein subunit, which C-terminus has a cysteine and a biotin tag; the amino acid sequence of the functional protein subunit is set forth in SEQ ID NO:1 or SEQ ID NO:2; the number of the dysfunctional protein subunit is not greater than 2, and the amino acid sequence is set forth in SEQ ID NO:3.
2) A streptavidin binds to the biotin tag of the scaffold protein. The streptavidin can bind 1 to 3 biotin-tagged antigens. The streptavidin may or may not have detectable labels.

Lastly, the present invention demonstrates the applications of the CD19 antigen-multimer, HER2 antigen-multimer, Tn antigen-multimer in the CAR staining, CAR expressing-cell specific expansion, and CAR expression detection.

The present invention demonstrates the applications of multimer and tetramer in the preparation of CAR staining reagent, CAR-expressing cell specific expansion reagent, and CAR expression detection reagent. The streptavidin contained in multimer or tetramer is used for binding to the biotinylated antigen that can specifically binds to target CAR.

To produce the high-affinity CAR staining reagent for CAR-T cell detection, we design, construct, and validate two compositions of antigen-multimers: antigen-streptavidin tetramer and antigen-dodecamer. The antigen-multimer comprises of streptavidin scaffold protein(s), streptavidin(s), and antigen(s) (FIG. 1A).

In all the experiments, the antigen is not randomly but site-specifically biotinylated, therefore reducing the clustering of CAR binding sites and steric hindrance. We validate the effectiveness of three different types of second-generation CAR: anti-CD19 CAR (clone FMC63, “Construction and Pre-clinical Evaluation of an Anti-CD19 Chimeric Antigen Receptor”, J Immunother. 2009 September; 32(7): 689-702. doi: 10.1097/CJI.0b013e3181ac6138), anti HER2-CAR (clone C6ML3-9, “T Cell Activation by Antibody-Like Immunoreceptors: Increase in Affinity of the Single-Chain Fragment Domain above Threshold Does Not Increase T Cell Activation against Antigen-Positive Target Cells but Decreases Selectivity”, J Immunol 2004; 173:7647-7653. doi: 10.4049/jimmunol.173.12.7647), and anti-Tn CAR (clone 237, “Multiple cancer-specific antigens are targeted by a chimeric antigen receptor on a single cancer cell”, JCI Insight. 2019;4(21): e130416. https://doi.org/10.1172/jci.insight.130416) (FIG. 1B). α

Each CAR used for validation from N terminus to C terminus comprises extracellular single chain antibody, CD8α hinge and transmembrane region, 4-1BB and CD3ζ intracellular domain, and monomeric enhanced green fluorescence protein (meGFP). The C terminus meGFP is used for tracking the CAR transduction efficiency and expression level. The design of hinge, transmembrane region and intracellular region is identical to that of Tisagenlecleucel CAR used in clinic. The anti-CD19 CAR and anti-HER2 CAR is human domain, while anti-Tn CAR is mouse domain. These three CARs used for validation can efficiently test the potency of the antigen-multimer: 1) the anti-CD19 CAR uses the identical FMC63-based single chain antibody fragment of that of CD19 CAR-T cell therapy in clinic; 2) The binding affinity K_{D} of CARs ranges from 0.3 nM to 140 nM, representing CAR with distinct binding affinity; 3) the molecular weight of antigens ranges from 2.6 kDa to 73 kDa.

The benefits of present invention:

According to the test results of cell lines and patient derived specimen, the antigen-multimer can highly specifically, highly sensitively, and highly precisely detect the CAR, can be used to detect rare CAR-T cells through magnetic particle enrichment, stimulate the specific phenotype and specifically expand these cell populations under the temperature control and specific CAR-T cell stimulation, and analyze the CAR-T cells multi-dimensionally through single cell multi-omics analysis. With alternative antigens, the antigen-multimer can be easily applied to the known CAR and used to discover new CAR. We validate the applications in three different CARS (anti-CD19, anti-HER2, and anti-Tn). Although we only test the antigen-tetramer and antigen-dodecamer in present study, the antigen-multimer can be extended to other valency combinations, such as dimer, pentamer, octamer and dextramer. Based on the multifunctionality and versatility, antigen-multimer is a class of novel CAR staining reagent, which can be applied to CAR-T cells, CAR-NK cells, CAR-macrophage, and other CAR-transduced cells and has broad applications in basic research and clinic. In addition, the methodology applying antigen-multimer to manufacture CAR-T cells, is different from the conventional protocol applying nonspecific anti-CD28/CD3 magnetic bead to stimulate and expand T cells. The antigen-multimer specifically stimulates and expands the respective CAR-T cells, without stimulating other T cells and inherent toxicity of magnetic beads, serves as a superior CAR-T manufacturing technology. In addition, the present invention provides the methodology of oligo nucleotide-labeled antigen-multimer to facilitate single cell sequencing and detection of CAR expression.

### BRIEF DESCRIPTION OF THE DRAWINGS

FIG. 1. Antigen-multimer design and validation in CARs directed against CD19, HER2, and Tn-glycoside. (FIG. 1A) Cartoon depicting antigen-tetramer and antigen-dodecamer with each major molecular component. (FIG. 1B) Diagram depicting CD19-, HER2-, and Tn-directed CAR constructs used to validate antigen-multimers. Each CAR contains a C-terminal meGFP to track CAR expression. CAR transcription is directed by either a spleen focus-forming virus (SFFV) or a murine embryonic stem cell virus (MSCV) promoter. (FIG. 1C, FIG. 1F, FIG. 1I) Staining titration of three types of antigen-tetramers (Tet) and antigen-dodecamers (Dod) on their matched CAR-transduced cell lines. Staining of the untransduced cell line is shown in gray and used for gating. Histograms are representative of three independent titrations. (FIG. 1D, FIG. 1G, FIG. 1J) Triplicate staining titration results for antigen tetramers, antigen dodecamers, and monomeric antigen controls were fitted to dose-response curves. The mean±standard error of the mean is depicted for each concentration. (FIG. 1E, FIG. 1H, FIG. 1K) Plots from a representative titration (fitted to dose-response curves) showing the relationship between geometric mean fluorescence intensity and staining reagent concentration on CAR-transduced (T) and untransduced cell lines (U).

FIG. 2. CAR transduction and BSA-multimers negative controls. (FIG. 2A, FIG. 2B, FIG. 2C) Histograms showing transduction of monomeric enhanced GFP-tagged CARs directed against CD19, HER2, and Tn-glycoside into human Jurkat cells (anti-CD19 and anti-HER2 CARs) or murine 58−/− hybridoma cells (anti-Tn CAR). Untransduced cells served as negative controls. (FIG. 2D, FIG. 2E) Histograms showing titration (from 0.1 to 10 nM) of Alexa 647-labeled bovine serum albumin-multimers (BSA-multimers) on untransduced and αCD19-CAR-transduced Jurkat cells. Gates to quantify non-specific staining percentage from the isotype control were established based on cells not stained with BSA-multimers (0 nM). (FIG. 2F) Staining titrations are performed on the respective CAR-transduced cell lines with CD19, HER2, or Tn PDPN antigen-monomer. The untransduced cells are shown in gray and used for gating. (FIG. 2G) The staining titrations of Tn-PDPN antigen-monomer at 1,000-fold higher concentration.

FIG. 3. Antigen-multimers are highly specific. (FIG. 3A, FIG. 3B) Antigen-tetramers (FIG. 3A) and antigen-dodecamers (FIG. 3B) were evaluated for specificity by staining cell lines transduced with CD19-, HER2-, and Tn-directed CARs. Staining of untransduced cell lines is shown in gray and used for gating. (FIG. 3C) Similar staining assays were conducted with existing CAR-staining reagents, which include polyclonal anti-IgG antibodies and Protein L. Staining of untransduced cell lines is shown in gray and used for gating. Histograms are representative of three independent titrations.

FIG. 4. Staining titrations of anti FMC63 antibody on αCD19 CAR-T cells. (FIG. 4A) Histograms showing titration of anti-idiotypic antibody (anti-FMC63) on αCD19-CAR-transduced Jurkat cells. Staining of the untransduced Jurkat cell line is used for gating. Histograms are representative of three independent titrations. (FIG. 4B) Triplicate staining titration results were fitted to dose-response curves. The mean±standard error of the mean is depicted for each concentration. (FIG. 4C) Plots from a representative titration (fitted to dose-response curves) showing the relationship between geometric mean fluorescence intensity and antibody concentration on CAR-transduced (T) and untransduced cell lines (U). (FIG. 4D, FIG. 4E) The staining titration results of anti-FMC63 antibody, CD19 antigen-tetramer, CD19 antigen-dodecamer are compared. The staining EC₅₀ are calculated from the dose-response curves (FIG. 4D) and the 1/EC₅₀ of each staining reagent are shown in bar graph (FIG. 4E). Fitted EC₅₀ values between antigen-tetramers and antigen-dodecamers were compared with a sum-of-squares F-test, whereby *** denotes p<0.001.

FIG. 5. Antigen-multimers are highly sensitive and precise. (FIG. 5A) Diagram depicting spike-in assays to measure a CAR-staining reagent's sensitivity and precision. Cell mixtures, constructed from CAR-meGFP-expressing cells and mCherry-expressing non-CAR cells, were stained with a CAR-staining reagent. After analysis by flow cytometry, the cells were placed into four categories: true positives (TP), true negatives (TN), false positives (FP), and false negatives (FN). Formulas to calculate prevalence, sensitivity, and precision are depicted. (FIG. 5B) Two anti-CD19 CAR-meGFP-transduced clones (“low clone” on top row and “mid clone” on bottom row) with low CAR expression were spiked into mCherry-expressing non-CAR cells at various proportions. Subsequently, CD19-tetramers (left) or CD19-dodecamers (right) were applied. Cells on the flow plots are colored according to fluorescent protein expression. (FIG. 5C, FIG. 5D) The prevalence of CAR cells (meGFP+) in cell mixtures plotted against the percentage of cells stained by CAR-staining reagents (left), sensitivity of detection (middle), and precision of detection (right) from spike-in assays with the low clone (FIG. 5C) and mid clone (FIG. 5D). For the stained percentage plot, if a point lies on the dotted line of unity, then the staining reagent accurately captured CAR cell prevalence. For the sensitivity and precision plots, if a point lies on the dotted line (100%), then the staining reagent was completely accurate in discriminating CAR cells from non-CAR cells.

FIG. 6. Sensitivity and precision of CAR-staining reagents. (FIG. 6A) Bar graph showing CAR expression (through meGFP fluorescence) in two anti-CD19 CAR-transduced clones (named “low clone” and “mid clone”) that were sorted for stably low CAR expression. The untransduced and pre-sort fluorescence are shown for comparison. CAR expression was compared by one-way ANOVA and post-hoc t-tests (Sidak multiple comparison correction), whereby * denotes p<0.05, ** denotes p<0.01, *** denotes p<0.001, and **** denotes p<0.0001. (FIG. 6B) Histograms showing expression of monomeric enhanced GFP-labeled CARs (top) and mCherry (bottom) on both CAR cell clones and the non-CAR cell clone. These three clones were used to quantify sensitivity and precision in cell mixtures. (FIG. 6C) Bar graph depicting the geometric average fluorescence intensity of untransduced, “low clone”, or “mid clone” stained with 3 nM of AF647-labeled antigen-multimers. (FIG. 6D) Line graph depicting the correlation of meGFP fluorescence with equivalent quantity of meGFP molecules. The dots are used to construct standard curve and measure the average CAR expression of “low clone”, “mid clone”, and unsorted polyclonal cells, as shown in bar graph. (FIG. 6E, FIG. 6G) meGFP+ anti-CD19 CAR cells (“low clone” on top row and “mid clone” on bottom row) were spiked into mCherry+ non-CAR cells at various proportions. Subsequently, monomeric CD19 (FIG. 6E), polyclonal anti-IgG (FIG. 6F) or Protein L (FIG. 6G) were applied to detect CAR cells by flow cytometry. CAR and non-CAR cells are colored according to the legend.

FIG. 7. Antigen-multimers magnetically enrich for CAR-T cells. (A) Cartoon depicting magnetic enrichment procedure. Mixtures of meGFP+ CAR cells and mCherry+ non-CAR cells were stained with either APC-labeled antigen-tetramers or antigen-dodecamers, followed by staining with anti-APC magnetic microbeads. The stained cell mixture (left) is applied to a magnetic column for negative (middle) and positive (right) selection. (B-D) Flow plots showing prevalence of meGFP+ CAR cells and mCherry+ non-CAR cells (“low clone” on top row and “mid clone” on bottom row) under magnetic enrichment with antigen-tetramers or antigen-dodecamers. The initial stained mixture, column wash, and column elution are shown in B, C, and D respectively.

FIG. 8. APC-labeled CD19-multimer staining of CAR-T cells from magnetic enrichment. (FIG. 8A, FIG. 8B) Histograms show APC-multimer staining of the cell mixture (“low clone” on top row and “mid clone” on bottom row) before (FIG. 8A) and after (FIG. 8B) magnetic enrichment with antigen-tetramers or antigen-dodecamers. Gates are established from a fluorescence-minus-one control.

FIG. 9. Antigen-multimers specifically stimulate CAR-T cells in a temperature-controlled manner. (FIG. 9A, FIG. 9B) Representative flow plots showing expression of CD69 (left) and CD25 (right) on anti-CD19 CAR-transduced cells after stimulation with anti-FMC63, CD19-tetramers, or CD19-dodecamers at various concentrations at 37° C. (top row) and 4° C. (bottom row). (FIG. 9C, FIG. 9D) Bar graphs depicting CD69 expression (left), CD25 expression (middle), and IL-2 secretion (right) of quadruplicate data from staining of anti-CD19 CAR-transduced cells at 37° C. (FIG. 9C) or 4° C. (FIG. 9D). The effects of CAR-staining reagents were compared by 2-way ANOVA and post-hoc t-tests, whereby ns denotes not significant, *denotes p<0.05, ** denotes p<0.01, *** denotes p<0.001, and ** * denotes p<0.0001.

FIG. 10. Antigen-multimer stimulation of 1st-gen CAR-T cells. (FIG. 10A, FIG. 10B) Representative flow plots showing expression of CD69 (FIG. 10A) and CD25 (FIG. 10B) on 1st-generation anti-CD19 CAR cells in response to stimulation from CD19-tetramers or CD19-dodecamers at various concentrations at 37° C. (FIG. 10C) Bar graphs depicting CD69 expression (left), CD25 expression (middle), and IL-2 secretion (right) of triplicate data from stimulation of anti-CD19 CAR cells. For all bar graphs, stimulation of CAR cells was compared by 2-way ANOVA and post-hoc t-tests, whereby ns denotes not significant, * denotes p<0.05, ** denotes p<0.01, *** denotes p<0.001, and **** denotes p<0.0001. (FIG. 10D) Bar graphs depicting the cell viability of CAR-transduced Jurkat cell line or patient-derived primary cells in response to stimulation from antigen-multimers.

FIG. 11. Antigen-multimers detect CAR-T cells from patient infusion product, peripheral blood, and tumor biopsies. (FIG. 11A) Diagram depicting the different patient biospecimens that may contain CAR-T cells throughout their ex vivo and in vivo life cycle. CAR-T cells are manufactured in the infusion product, circulate through peripheral blood, and home into the tumor. (FIG. 11B) Representative flow plots showing that CD19-tetramers (top row) and CD19-dodecamers (bottom row) can detect both CD4⁺and CD8⁺CAR-T cells from axicabtagene ciloleucel (axi-cel) and tisagenlecleucel (tisa-cel) infusion products. Flow plots are representative of six independent infusion products (3 axi-cel and 3 tisa-cel). Gates were drawn off CD19-multimer fluorescence-minus-one (FMO) controls. (FIG. 11C) Relative percentage of maximum staining from infusion product titrations were fitted to dose-response curves. Since each infusion product contains a different prevalence of CAR-T cells, percentage stained was normalized to that of maximum staining for each infusion product. For each concentration, the point displays mean±standard error of the mean. The fitted staining EC₅₀ values were compared with a sum-of-squares F-test. (FIG. 11D, FIG. 11E) Flow plots showing CD19-tetramer staining of a longitudinal set of peripheral blood samples from a patient with diffuse large B-cell lymphoma infused with an axi-cel infusion product. CAR-T cells were analyzed for CD4 and CD8α expression. Expansion and contraction of CAR-T cells is summarized in (FIG. 11E). (FIG. 11F) Staining of a dissociated lymphoma tumor biopsy from a patient treated with an axi-cel infusion product. CD3⁺CAR⁺ cells were analyzed for CD4 and CD8α expression.

FIG. 12. Antigen-multimers isolate CAR-T cells from a patient biospecimen for single-cell omics analyses. (FIG. 12A) Flow plots demonstrating use of CD19-tetramers for CAR-T cell sorting and single-cell, omics assays. After gating for CD3⁺ T cells, PBMCs from a healthy donor (biological control) were used to draw both CAR− and CAR+ gate. Gates were used to sort for endogenous CAR− T cells (Endo-T) and therapeutic CAR+ T cells (CAR-T) from a patient biospecimen. Sorted Endo-T and CAR-T cells were used for single-cell omics (RNA-seq and TCR-seq). (FIG. 12B) Violin plot depicting normalized CAR transgene mRNA expression in Endo-T and CAR-T samples. (FIG. 12C) Pie chart depicting distribution of TCR clonotypes between Endo-T and CAR-T cells. (FIG. 12D) Single-cell data from Endo-T and CAR-T were combined and visualized by UMAP and unsupervised Louvain clustering. Twelve T-cell clusters were annotated based on known markers. (FIG. 12E) Stacked bar graph depicting proportions of Endo-T and CAR-T cells represented in each T-cell cluster. (FIG. 12F) Volcano plot depicting differentially expressed genes (DEGs) between Endo-T and CAR-T cells, with a cut-off based on log 2 fold-change. DEGs of interest are labeled. Abbreviations: cyto, cytotoxic; CM, central memory; EM, effector memory; TE, terminal effector.

FIG. 13. CAR transgene distribution, cell cluster annotation, and TCR clone size distribution of single-cell omics data. (FIG. 13A) UMAP depicting the sample origin of each cell in the single-cell dataset. (FIG. 13B) UMAP depicting normalized expression of the CAR transgene in each cell. (FIG. 13C) UMAP depicting all sixteen T cell and non-T cell clusters. (FIG. 13D) Dot plot depicting expression of key genes used to annotate T cell and non-T cell clusters. (FIG. 13E) Dot plot depicting expression of key genes used to annotate T-cell subsets. (FIG. 13F) UMAP depicting computational cell cycle analysis of cells. (FIG. 13G) Stacked bar graph depicting CD4⁺ versus CD8⁺ T-cell distribution within Endo-T and CAR-T samples. (FIG. 13H) Heatmap showing TCR clone size distributions in Endo-T and CAR-T samples. Most TCR clones are specific to CAR-T (top row) or Endo-T (left column) samples. Remaining TCR clones were discovered in both samples and are plotted according to their clone sizes in CAR-T and Endo-T samples.

FIG. 14. Antigen-multimers detect CAR-transduced NK-92 cells. NK-92 cells are transduced with 4-1BB-based second-generation CAR with the transduction efficiency of ˜2% and labeled with meGFP. The cells are stained with CD19 antigen-multimers. Flow plots depicting the staining results of negative control (Left), antigen-tetramers (Middle), and antigen-dodecamers (Right, 5 nM).

FIG. 15. Oligo nucleotide-labeled antigen-multimers detect patient-derived CAR-T cells. Histogram depicting the single cell expression of CAR expressed on the cell surface of peripheral blood cells (pre-CAR transduction, Aphe) and infusion product (post-CAR transduction, IP) of two diffuse large-B lymphoma patients subject to CAR-T cell therapy. The samples are stained with oligo nucleotide-labeled CD19 antigen-multimers (5 nM). The CAR expression is detected with single cell sequencing.

FIG. 16 Expansion of CAR-T cells. (FIG. 16A) Bar graphs depicting the fold expansion of CAR-T cells incubated with IL-2, and under the treatment of no multimers, 5 nM of antigen-tetramers, or 5 nM of antigen-dodecamers whereby *** denotes p<0.001, **** denotes p<0.0001, and ns denotes no significance. (FIG. 16B) Flow plots depicting the fluorescence intensity of meGFP (x-axis) and SSC (y-axis) of meGFP-labeled CAR-transduced human primary T cell populations. The untransduced control cells are used for gating of CAR+ T cells.

FIG. 17. The expression of activation markers (CD69/CD25). Flow plots depicting the expression of CD69 (x-axis) and CD25 (y-axis) of meGFP-labeled CAR-transduced human primary T cell populations under the treatment of antigen-multimers. Gates are established from a fluorescence-minus-one control.

FIG. 18 The expression of PD-1. Flow plots depicting the expression of PD-1 of meGFP-labeled CAR-transduced human primary T cell populations under the treatment of antigen-multimers. Gates are established from a fluorescence-minus-one control.

FIG. 19 Expansion of CAR-T cells under modified conditions. Bar graphs depicting the fold expansion of CAR-T cells incubated with IL-2, and under the treatment of no multimers, culture surface-coated 5 nM of antigen-tetramers, or culture surface-coated 5 nM of antigen-dodecamers whereby *** denotes p<0.001.

### DETAILED DESCRIPTION

The present invention is illustrated in detail with the embodiments and FIG.s. The applied multimer is in the form of dodecamer or streptavidin tetramer. The structure and property of multimer are disclosed in the China Patent application “One Multimeric Binding Reagents” (CN107847544A). The complex of dodecamer or streptavidin tetramer binding with respective antigens is called antigen-dodecamer or antigen-streptavidin tetramer (in principle, both belong to antigen-multimer). Other materials used in the embodiments are commercially available.

### Embodiment 1: Production of Antigen-Multimers

The antigen portion of antigen-multimer can be biotin-labeled human CD19 polypeptide fragment (SEQ ID No. 4), biotin-labeled human HER2 polypeptide fragment (SEQ ID No. 5), biotin-labeled Tn glycoside podplanin G (T*) KPPLEE polypeptide (SEQ ID No. 6, the * is glycosylation tag), or biotin-labeled bovine serum albumin (BioVision, 7097-5). The above-mentioned human CD19 and HER2 polypeptides comprise of an amino acid sequence that can be biotinylated (SEQ ID No.7: GLNDIFEAQKIEWHEKLGLEVLFQGPELEHHHHHHHHHH*)

The scaffold subunit of multimer can be Alexa Fluro 647-labeled streptavidin (BioLegend, 405237) or allophycocyanin-labeled streptavidin (BioLegend, 405207).

To produce antigen-streptavidin tetramer, 4:1 molar ratio of biotin-labeled antigen is added to fluorescence-labeled streptavidin tetramer. The mixture is incubated at dark for 30 minutes at 4° C. The product is diluted with PBS to optimal concentrations for staining.

To produce antigen-dodecamer, 1:4 molar ratio of biotin-labeled scaffold subunit is added to fluorescence-labeled streptavidin tetramer. The mixture is incubated at dark for 30 minutes at 4° C. Then 12:1 molar ratio of biotin-labeled antigen is added and incubated at dark for 30 minutes at 4° C. The product is diluted with PBS to optimal concentrations for staining.

### Embodiment 2: Staining of CD19, HER2, or Tn Glycoside Antigen-Multimer

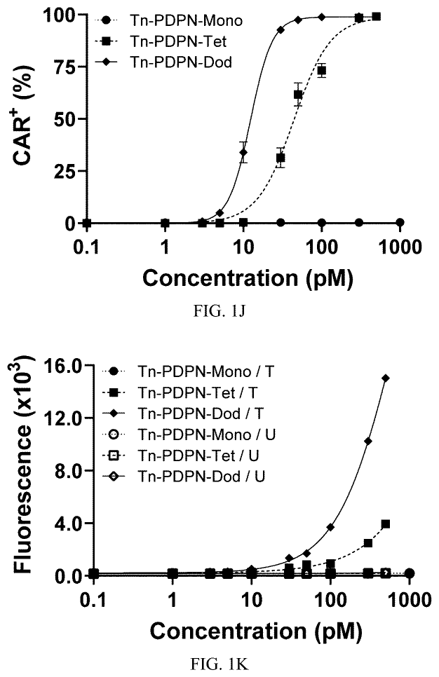

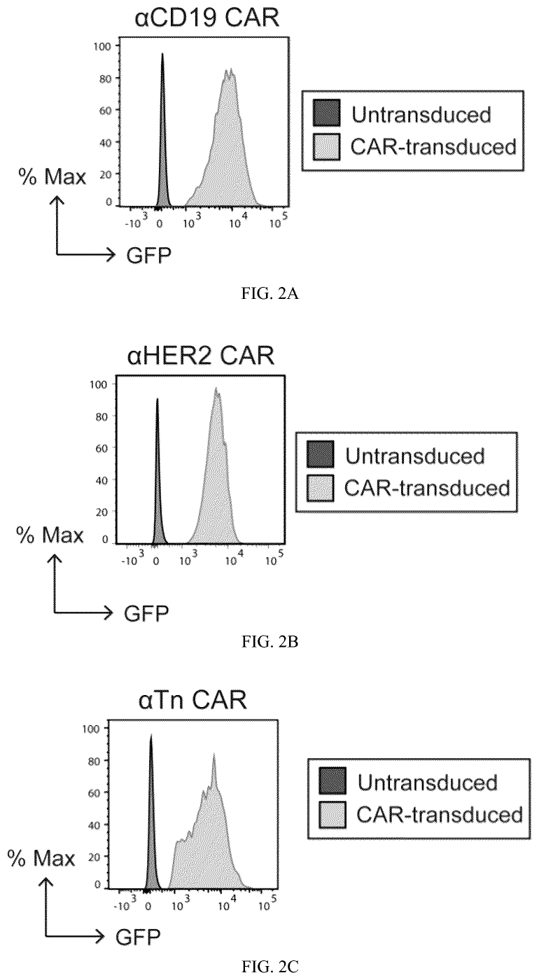

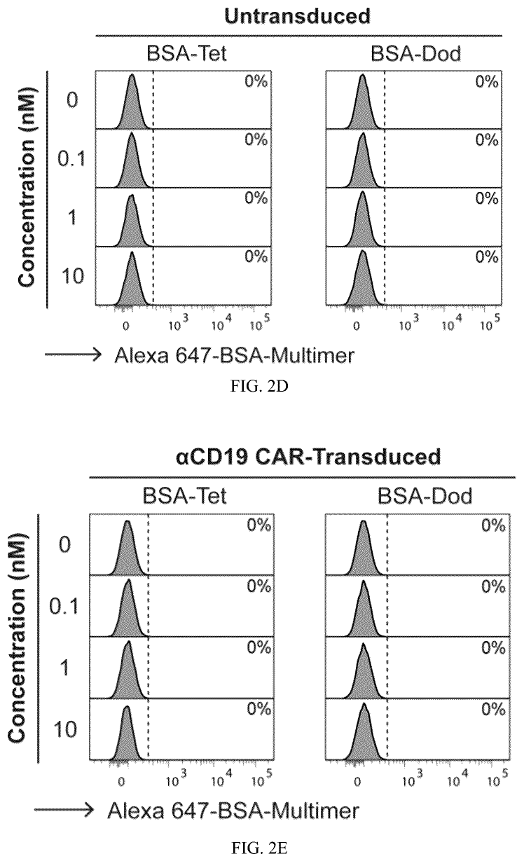

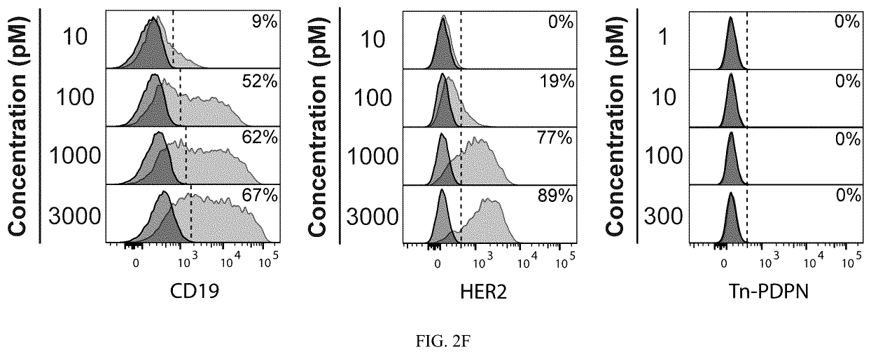

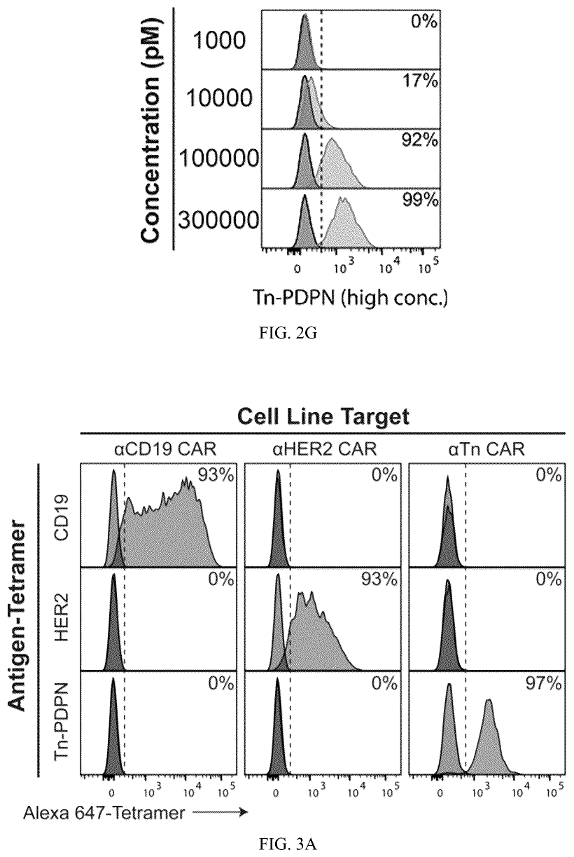

Firstly, the staining is validated in the CAR-transduced cell lines, including human Jurkat cell line and mouse 58^{-/-} hybridoma. The meGFP fluorescence tag of CAR serves as the detection maker of CAR transduction and expression (FIG. 2A-C). The titration staining is performed with CD19 antigen-multimer, HER2 antigen-multimer, and Tn-PDPN peptide-multimer. The antigen monomer is used as the control. The expression is detected by quantitating the fluorescence of meGFP with flow cytometry.

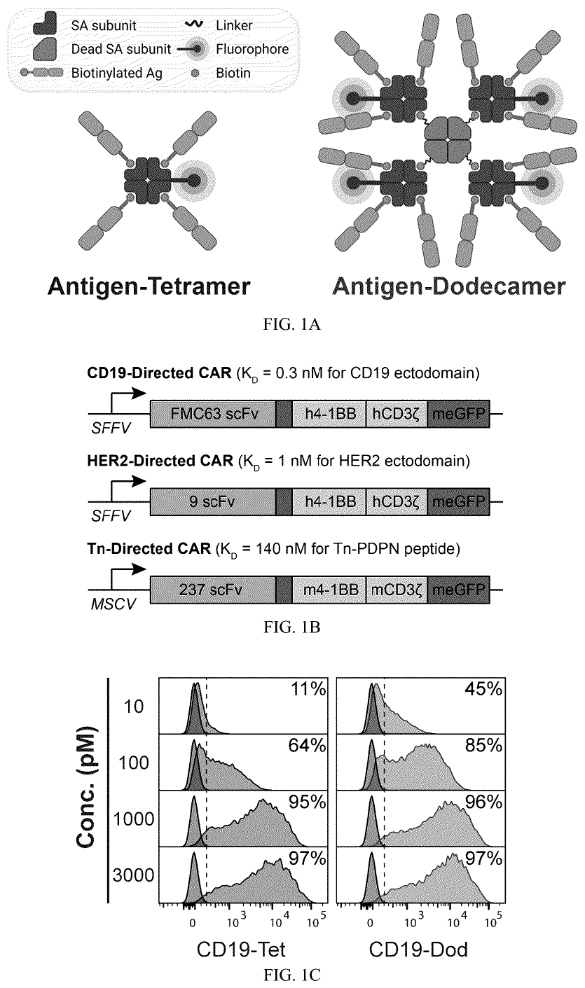

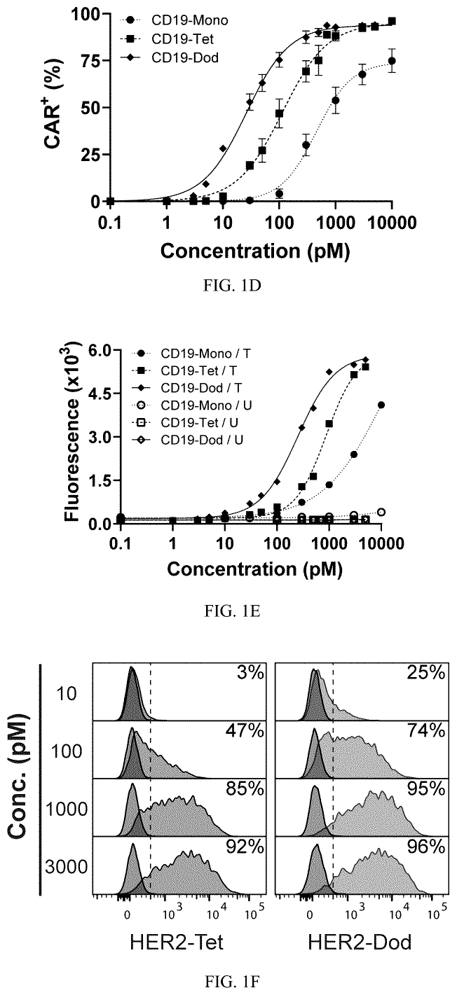

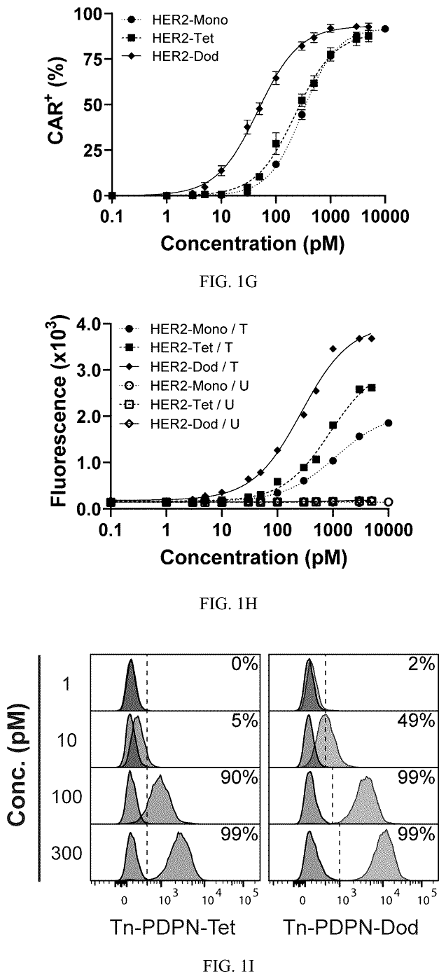

The titration staining experiment illustrates that the staining results of CAR-expressing cell lines stained with respective CD19 antigen-multimer, HER2 antigen-multimer, Tn-PDPN peptide-multimer (FIG. 1C, 1F, 1I). The cells without CAR expression are unstained. The negative control of bovine serum albumin (BSA)-multimer has negligible non-specific staining (FIG. 2D-E). With higher concentrations of antigen-multimer applied, the proportions of detected cell populations are higher (FIG. 1D, 1G, 1J). At the maximum concentration, more than 92% of target cells are detected by antigen-streptavidin tetramer or antigen-dodecamer. With higher concentrations of antigen-multimer applied, the average fluorescence intensities are increased (FIG. 1E, 1H, 1K). These findings demonstrate that the dose-dependent binding mode of CAR with the high affinity antigen-multimer. The non-specific background staining is negligible (shown in the BSA-multimer control).

Compared to antigen monomer, the two forms of antigen-multimer both intensify the fluorescence signal. Under the same molar concentrations, the fluorescence intensity of antigen-dodecamer is stronger than that of antigen-streptavidin tetramer. The magnitude of increased intensity is correlated to the K_{D} value of the CAR to respective antigen.

The magnitude of the increased intensity of high affinity CAR is relatively small, demonstrated by the staining of CD19 CAR (K_{D}=0.3 nM).

The magnitude of the increased intensity of medium affinity CAR is medium, demonstrated by the staining of HER2 CAR (K_{D}=1 nM).

The magnitude of the increased intensity of low affinity CAR is relatively large, demonstrated by the staining of Tn CAR (K_{D}=140 nM).

Specifically for Tn-PDPN peptide ligand, 4-fold higher fluorescence is detected in Tn-PDPN-dodecamer, compared to Tn-PDPN-streptavidin tetramer, while the staining is undetectable with Tn-PDPN-monomer at corresponding concentrations (FIG. 1K). The staining of antigen-monomer is detectable only at 1000-fold higher of concentration (FIG. 2G).

These experimental results demonstrate that only antigen-multimer can detect low affinity CAR (K_{D}>10 nM). One exemplary is the Tn CAR (K_{D}=140 nM). At normal concentrations, the staining can only be detected with antigen-multimer but cannot be detected with antigen-monomer. For the CAR with higher affinity, such as CD19 CAR (0.3 nM), the efficiency of detection with antigen-multimer is 25% higher than antigen-monomer (FIG. 1D). The portion of cell population detected by antigen-multimer but not antigen-monomer represents the sub-population of CAR-T cells with lower CAR expression. Overall, the higher binding avidity of antigen-multimer effectively augments the staining and detection of CAR, which is especially significant in low affinity CAR. In clinical cell therapy practice, following the infusion of CAR-T to the human body and the onset of recognition and attack of tumor cells, the expression of CAR will dramatically down-regulate. Therefore, it is critical to detect these low CAR-expressing CAR-T cell population in the clinical diagnosis. Meanwhile, the recent findings in literature show that low affinity CAR-T is superior to high affinity CAR-T in respect of effect of attacking tumor cells. However, only antigen-multimer can efficiently detect low affinity CAR. Taken together, the invented antigen-multimer is an innovative, unique, efficient, and exclusive CAR detection reagent, for both high affinity and low affinity CAR.

### Embodiment 3: The Antigen-Multimer Is Highly Specific

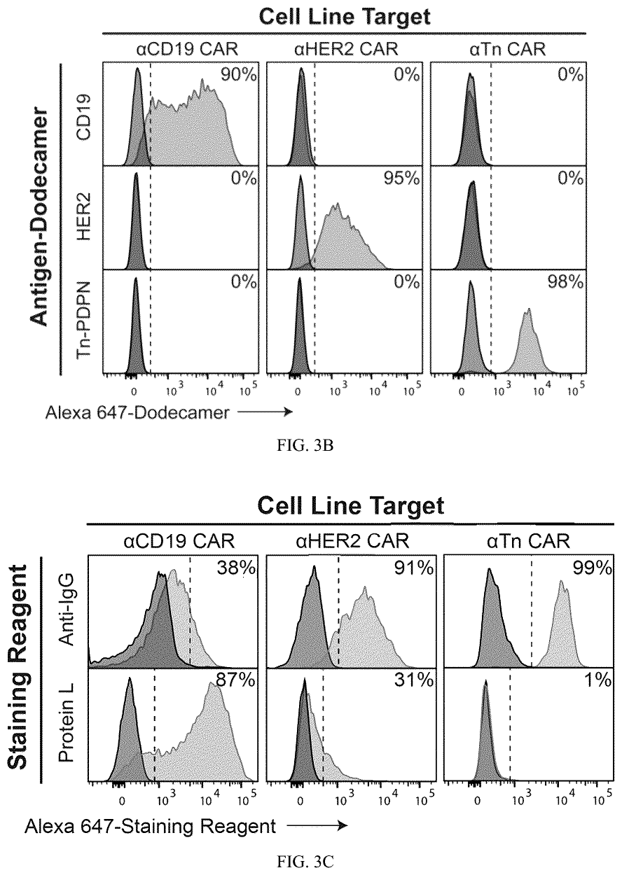

The specificity of staining is validated using three different antigen-streptavidin tetramer and antigen-dodecamer to stain the respective CAR-expressing cells. One of the non-specific controls is the cell without CAR expression, other non-specific controls include the cell expressing un-matched CAR. For example, the CD19 antigen-multimer should not be able to stain the HER2 or Tn CAR-expressing cells. The staining results show that antigen-streptavidin tetramer (FIG. 3A) and antigen-dodecamer (FIG. 3B) can detect ≥90% of respective CAR-expressing cells, while stain ≤1% of cells without CAR expression or cells expressing un-matched CARs. Therefore, the antigen-multimer is highly CAR-specific.

Meanwhile, the staining specificity of two conventional CAR staining reagents are evaluated: polyclonal IgG antibody (Fisher Scientific, A21237) and Protein L (Thermo Fisher Scientific, 29997). The underlying mechanism of both CAR staining reagents is through the binding to the Fab-like molecules on the cell surface, which cannot distinguish different CAR. Due to non-specific binding to the un-transduced cells without CAR expression, the staining efficiency of anti IgG antibody is significantly lower than that of CD19 antigen-multimer for the staining of CD19 CAR (FIG. 3C, top). The Protein L, which binds to immunoglobin Kappa light chain, can stain CD19 CAR (FIG. 3C, bottom), but can hardly stain HER2 CAR, and totally cannot stain Tn CAR. The observed inconsistency among different CAR staining may be due to the different affinity of Protein L to alternative Kappa light chain. These findings validate the conclusion that antigen-multimer is more specific than conventional CAR staining reagents.

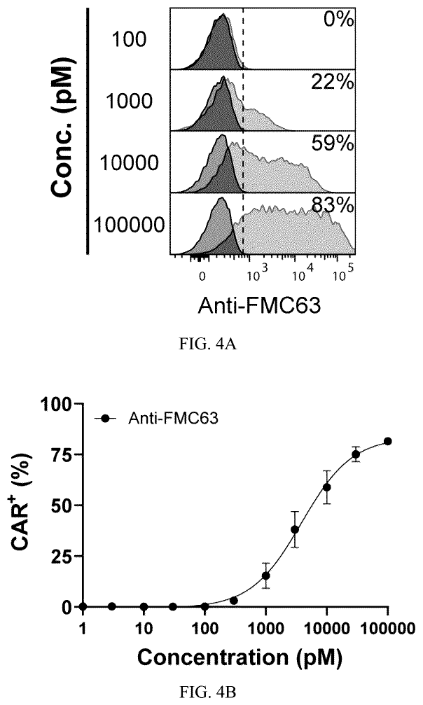

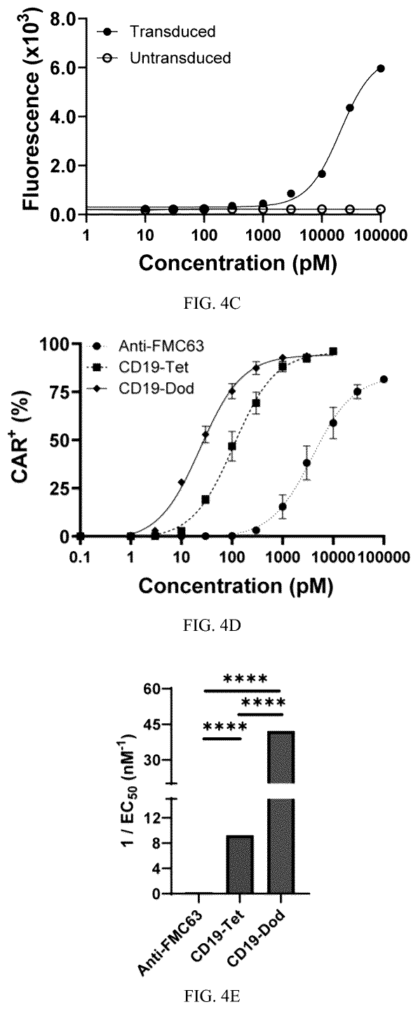

Lastly, the specificity of the only one commercially available FMC63 anti-idiotypic antibody is evaluated. This antibody is developed to detect FMC63-based CD19 CAR. However, in the study it is found that the antibody is not able to stain all the CD19 CAR. At the maximum concentration the anti-FMC63 antibody can detect 83% of CD19 CAR-expressing cells (FIG. 4A-B). This value is lower than that can be achieved with CD19 antigen-streptavidin tetramer (93%) and CD19 antigen-dodecamer (90%) at saturated concentrations. The non-specific staining of cells without CAR expression is negligible (FIG. 4C). The staining efficiency of CD19 antigen-streptavidin tetramer is about 8-fold higher, and the staining efficiency of CD19 antigen-dodecamer is about 40-fold higher, in comparison to anti FMC63 antibody (FIG. 4E). These finding demonstrate that although the anti-FMC63 antibody has the similar binding specificity as antigen-multimer to CD19 CAR, the staining efficiency is lower even at saturated concentrations (FIG. 4D-E). The observed lower binding efficiency may be due to the lower binding affinity to anti-CD19 CAR.

### Embodiment 4: The Antigen-Multimer Is Highly Sensitive and Highly Precise

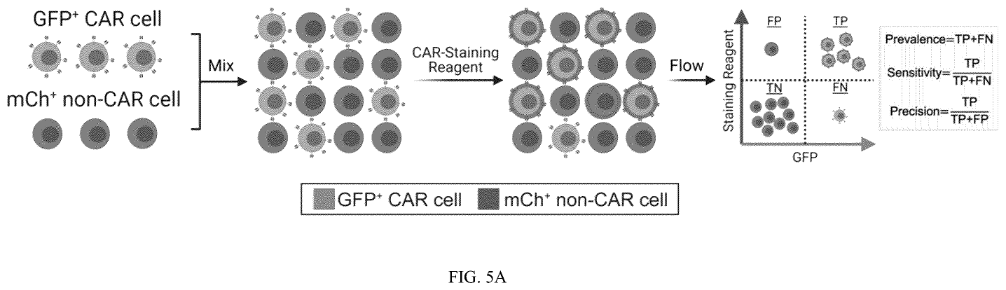

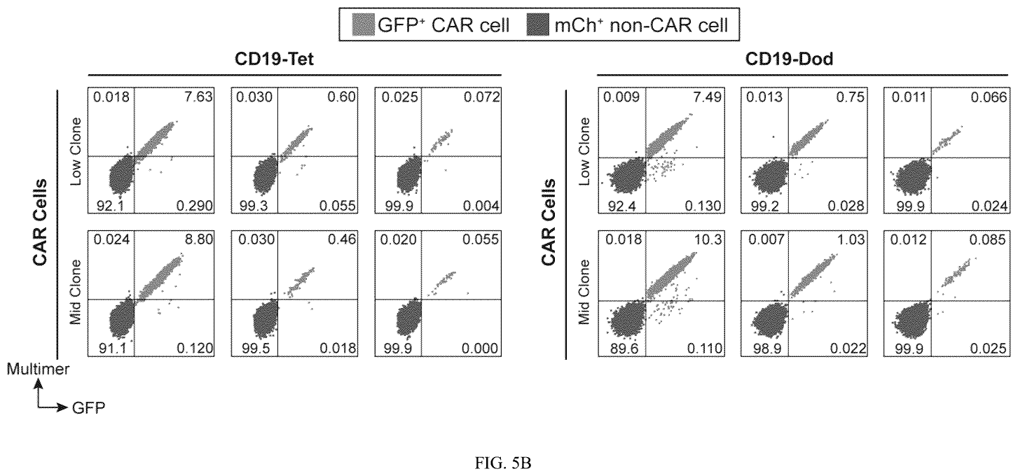

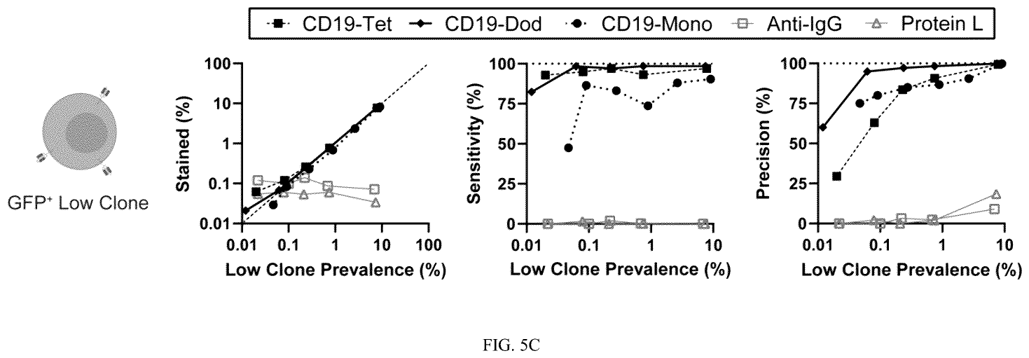

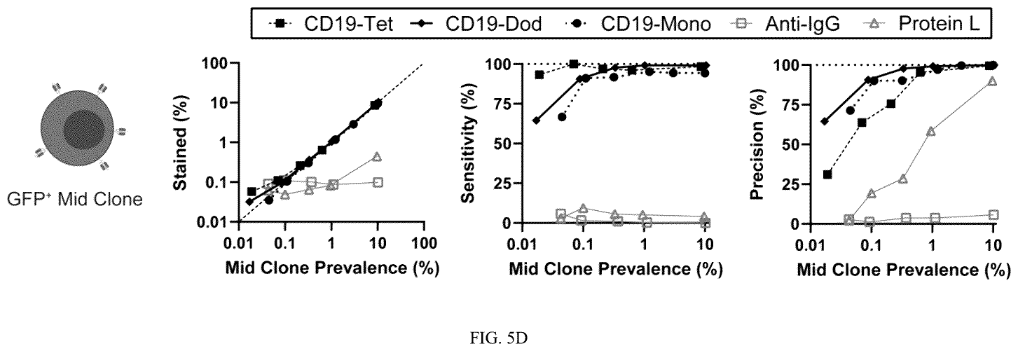

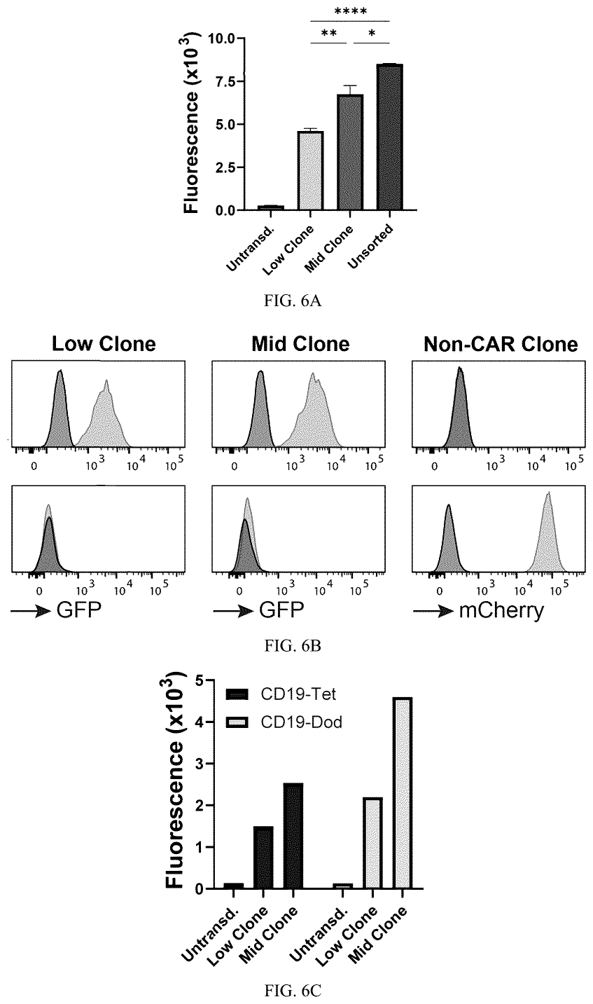

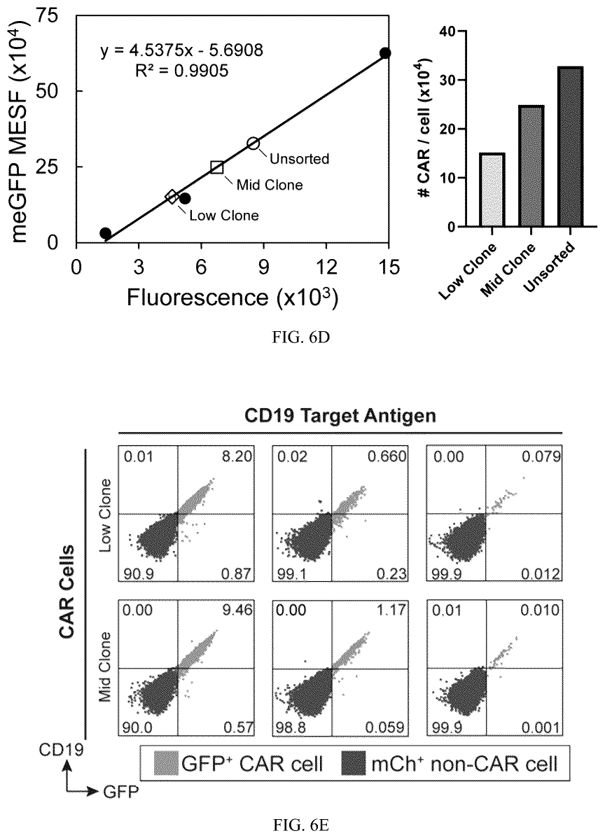

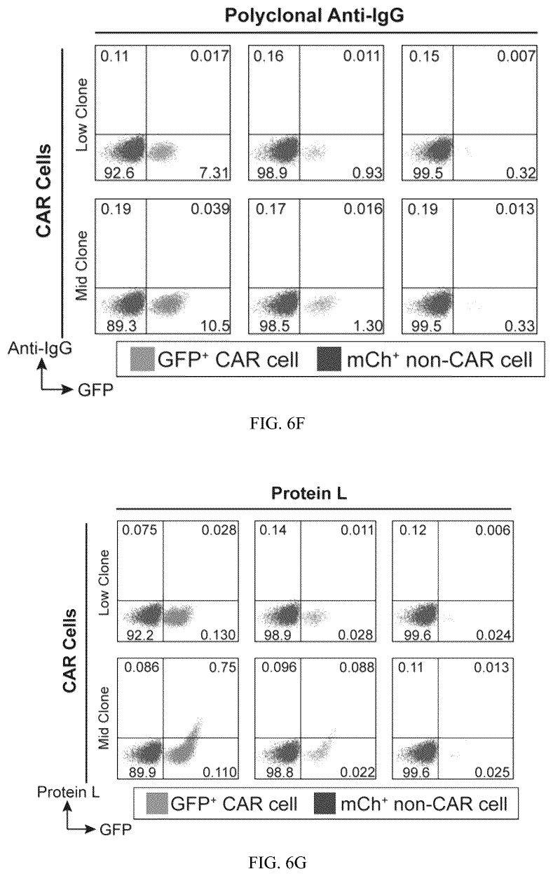

Following the validation of specificity, the sensitivity and precision of antigen-multimer is evaluated. The mixture of mCherry-labeled cells without CAR expression and meGFP-labeled CD19 CAR cells are stained with CD19 antigen-multimer and analyzed with flow cytometry (FIG. 5A). The sensitivity is measured by monitoring the false positivity. The sensitivity and precision are meaningful in practice, especially when the CAR-expressing cells are rare, and the expression level of CAR is low due to the down-regulated CAR expression following the binding of CAR to antigen. To mimic this scenario, the cells without CAR expression are incorporated into two monoclonal cell populations which stably expressed low levels of CAR, namely “Low Clone” or “Mid Clone”. The staining experiments are performed (FIG. 6A-D). In “Low Clone” cell population, each cell expresses an average of 150,000 CAR, and in “Mid Clone” cell population, each cell expresses an average of 250,000 CAR. The expression levels of both cell populations are less than the previously mentioned un-sorted polyclonal cell population, which express about 330,000 CAR per cell. The cells without CAR expression are transfected and labeled with mCherry.

Small portion of CAR-expressing cells are mixed with larger portion of cells without CAR expression. The ratio of CAR-expressing cells is decreased from 10% to 0.01%. The true-positive detection is measured with the percentage of GFP-positive cells that are detected by antigen-multimer (FIG. 5B). As predicted, “Mid Clone” has stronger staining than “Low Clone” (FIG. 6C). The CD19 antigen-multimer can detect as low as less than 0.1% incorporated CAR-expressing cells, with a sensitivity ≥90% (FIG. 5C-D). The precision of detection is different for the antigen-streptavidin tetramer and antigen-dodecamer when less CAR-expressing cells are incorporated. For the “Mid Clone”, when less than 1% of CAR-expressing cells are incorporated, the cells detected by antigen-dodecamer are purer than that detected by antigen-streptavidin tetramer. For “Low Clone”, when less than 10% of CAR-expressing cells are incorporated, the cells detected by antigen-dodecamer are purer than that detected by antigen-streptavidin tetramer. These finding demonstrate that CD19 antigen-multimer has high sensitivity and precision. Most importantly, even under the condition that the CAR expression level is low and the proportion of CAR-expressing cells is small (≤1%), CD19 multimer is able to sensitively detect and isolate CAR-expressing cells with high purity (≥90%) form the mixture of cell population.

Meanwhile, the conventional CAR staining reagents, including CD19 antigen-monomer, polyclonal anti IgG antibody and Protein L, are used to perform the detection experiments. For “Mid Clone”, the staining efficiency of CD19 antigen-monomer is comparable with that of CD19 antigen-multimer. However, the staining efficiency of CD19 antigen-monomer is lower than that of antigen-multimer in the detection of “Low Clone”. These findings illustrate that the higher binding avidity of CD19 antigen-multimer can increase the detection sensitivity to cells expressing low level of CAR. The anti IgG antibody fails to detect the cells expressing low level of CAR. For different ratios of incorporation, the detection sensitivity and precision are ≤10%. Protein L can detect some “Mid Clone” cells but cannot detect “Low Clone” cells. When “Mid Clone” cells are incorporated at the proportion of 10%, 90% of the cells can be detected. The sensitivity is greatly decreased to less than 10% when less CAR-expressing cells are incorporated. These findings demonstrate that neither anti IgG antibody nor Protein L can efficiently detect CAR-T cells when the CAR is expressed at low level per cell.

### Embodiment 5: CAR-T Cells Are Magnetically Enriched With Antigen-Multimer

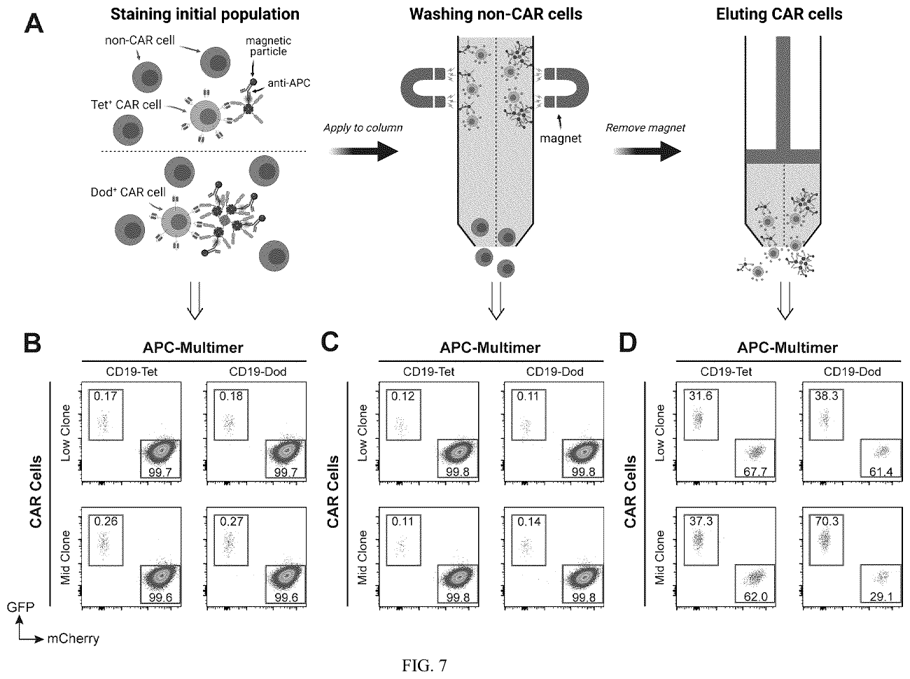

We predict that antigen-multimer can be applied to enrich rare CAR-T cells from cell mixture. To test this hypothesis, the CAR-expressing cells are magnetically selected with the antigen-multimer, and magnetic particle conjugated antibody (FIG. 7A). The CAR-T cells are stained with allophycocyanin (APC)-labeled CD19 antigen-streptavidin tetramer or CD19 antigen-dodecamer, followed by incubation with magnetic particle conjugated anti-APC antibody. The stained cells are loaded to magnetic column. The cells without CAR expression cannot attach to the column and are thus washed out (negative selection). After the removal of magnetic field, the retained CAR cells are eluted and collected (positive selection).

The CAR cells are enriched from the mixture of mCherry⁺ non-CAR cells and meGFP. CAR-expressing cells with the magnetic selection. To mimic the conditions that are challenging for cell enrichment, the CAR⁺ cells stably expressing low level CAR are applied (Low Clone and Mid Clone, as shown in FIG. 6A-C). The CAR⁺ cells are rare in the initial unsorted cell population (about 0.2%, FIG. 7B). According to the detection by flow cytometry of the CAR⁺ cells, about 35% of Low Clone and about 55% of Mid Clone cells are retained in the column (FIG. 7B and 7C). Following the negative selection, the eluted cells achieved more than 100-fold of enrichment (FIG. 7D).

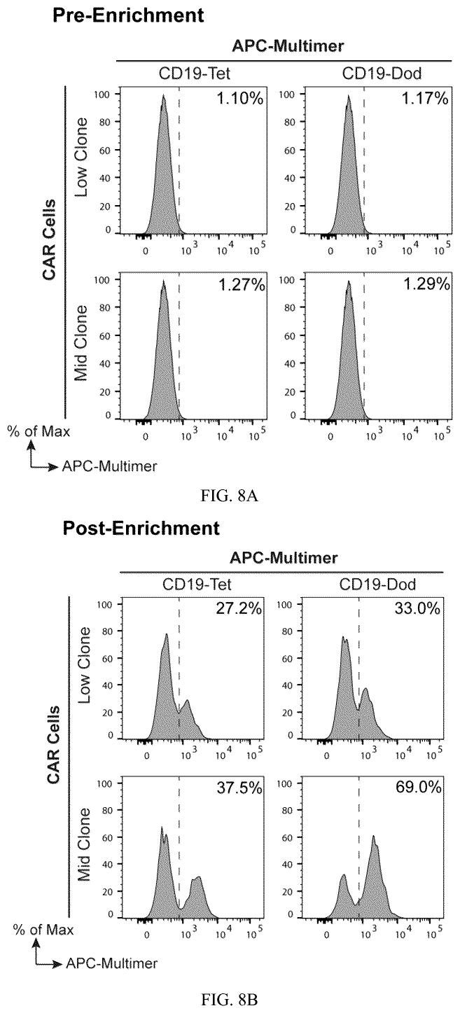

In addition, the APC fluorescence intensity of eluted cells is significantly higher than that of the initial stained cell population (FIG. 8). Compared to antigen-tetramer, the enrichment efficiency of the antigen-dodecamer is higher (FIG. 8B). The observation is consistent with the prediction since the staining intensity of dodecamer is stronger than that of tetramer. These findings demonstrated that antigen-multimer can enrich the low CAR-expressing and rare CAR-T cells. Post the enrichment with antigen-multimer and magnetic column, the proportion of CAR⁺ Cells increased more than 100-fold compared to that of pre-enrichment cell population.

### Embodiment 6: CAR-T Cells Are Specifically Stimulated by Antigen-Multimers With Temperature Control

The experiments are performed to validate whether the antigen-multimer can stimulate CAR-T cells after binding to the CAR on the cell surface. The specific stimulation of CAR can be measured with the activated phenotype, which is associated with the efficacy of CAR-T cells in the body. Conventionally, the T cells are stimulated with the T cell mitogens, such as anti-CD3/CD28 antibodies, phorbol 12 myristic acid 13 acetate (PMA), ionomycin, and phytohemagglutinin (PHA). Unfortunately, these mitogens are not CAR-expressing cell specific, and are not related to immune stimulation. Based on the high specific and high affinity binding of antigen-multimer with CAR, it is speculated that antigen-multimer can be applied to directly stimulate CAR-T cells in solution.

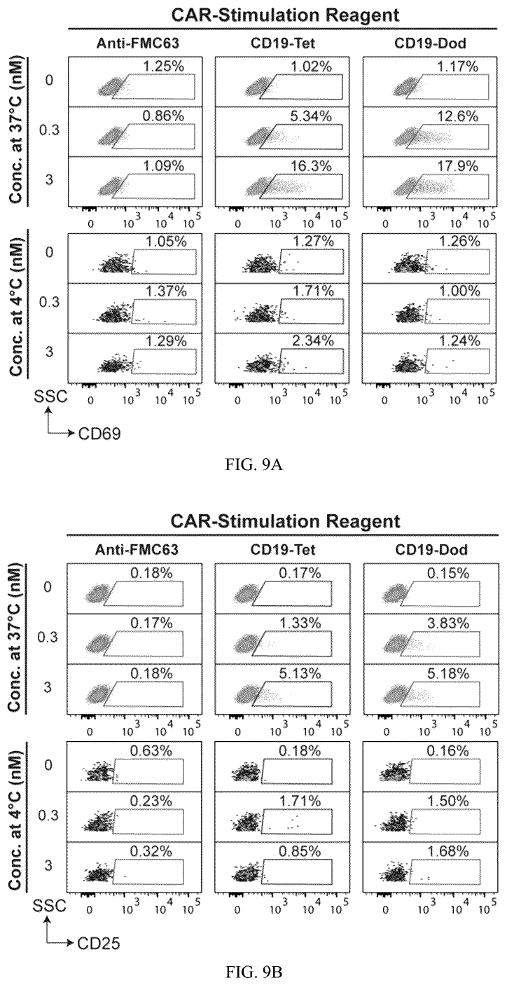

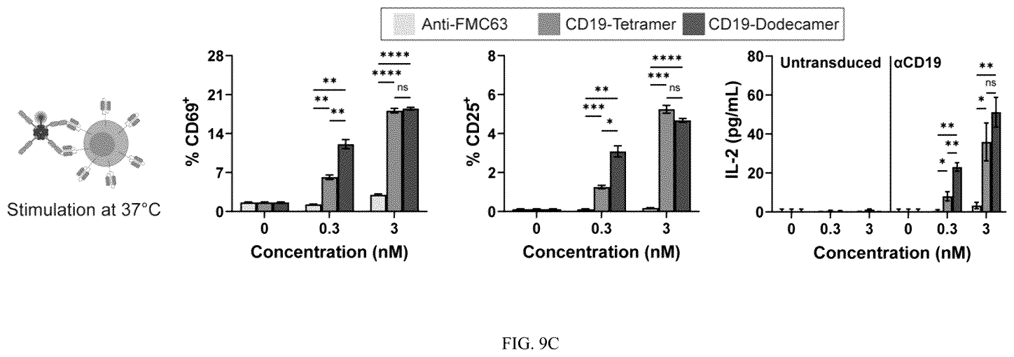

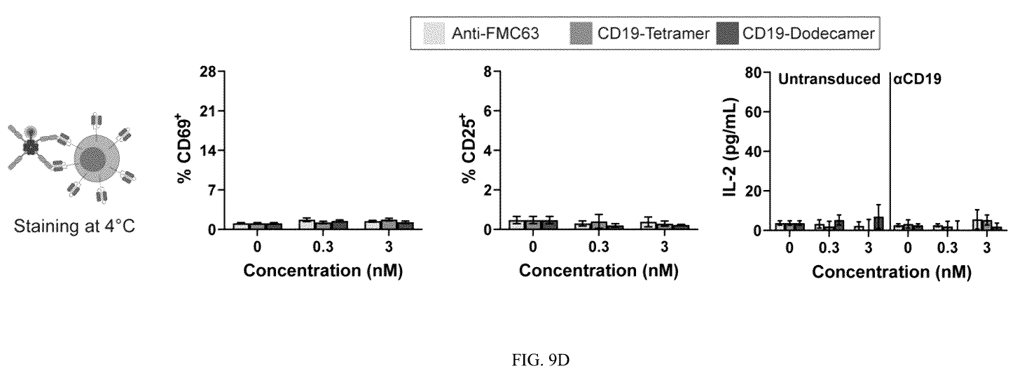

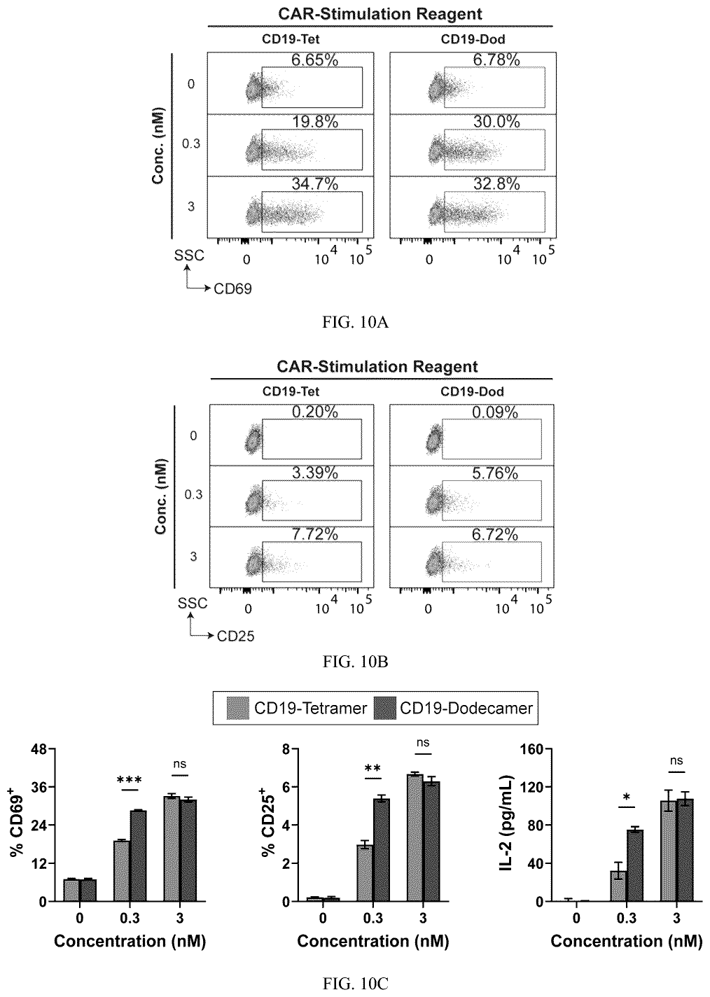

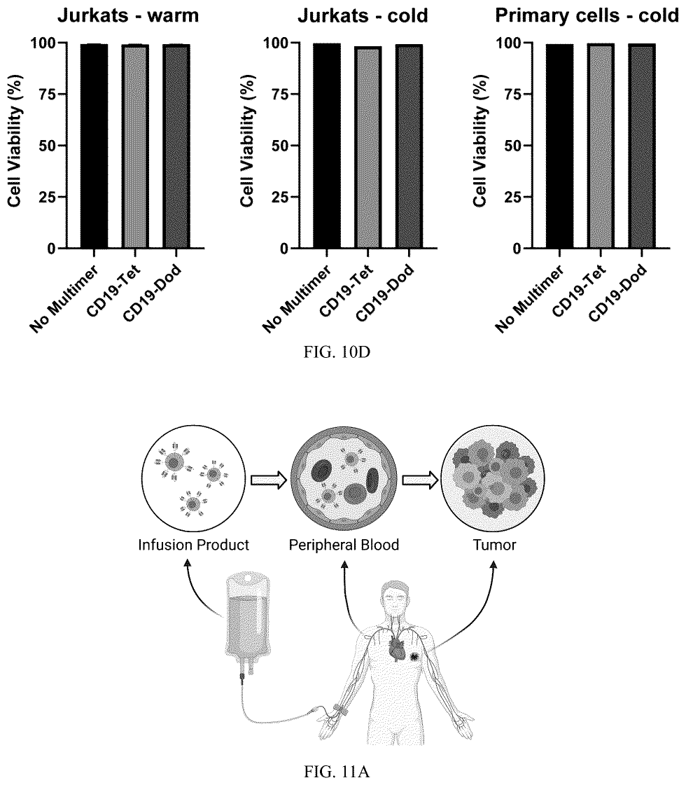

The second-generation anti-CD19 CAR-expressing cells are incubated with CD19 antigen-streptavidin tetramer or CD19 antigen-dodecamer at 37° C. It is found that the CD69 expression (early activation marker, FIG. 9A) and CD25 expression (late activation marker, FIG. 9B) are up regulated. With the increased multimer concentrations, the percentiles of CD69⁺ and CD25⁺ cells are increased (FIG. 9C). On the other hand, although anti FMC63 antibody can stain the CAR, it cannot activate the CAR-T cells at any concentrations tested in solution. The CAR cells stimulated with multimer secret the cytokine of IL-2, which is related to the up regulation of CD69 and CD25. At the concentration of 0.3 nM, the dodecamer treatment can promote higher IL-2 secretion compared to tetramer. At the concentration of 3 nM, the stimulation effects are comparable. Similar results are observed in the first-generation anti CD19 CAR-expressing cells (FIG. 10A-C). The tetramer or dodecamer treatment has no effect on the cell viability (FIG. 10D). These findings demonstrates that antigen-multimer can activate CAR-T cells at 37° C. solution, while anti-FMC63 antibody cannot activate CAR-T cells, likely due to the low binding avidity.

The CAR-expressing cells are incubated with CD19 antigen-multimer under the same conditions but at 4° C. (FIG. 9D). It is speculated that the metabolism can be slow down at low temperature, and the CAR stimulation signals can thus be attenuated. As expected, the up regulation of CD69 or CD25 was not observed under the treatment with any concentrations tested at 4° C., the IL-2 secretion is not detectable, and the cell viability is not affected (FIG. 10D). Therefore, the antigen-multimer cannot stimulate CAR cells at low temperature. These findings demonstrates that the staining can be performed at 4° C. to avoid unwanted stimulation.

### Embodiment 7: Detection of CAR-T Cells From Patient Infusion Product, Peripheral Blood, and Tumor Biopsy

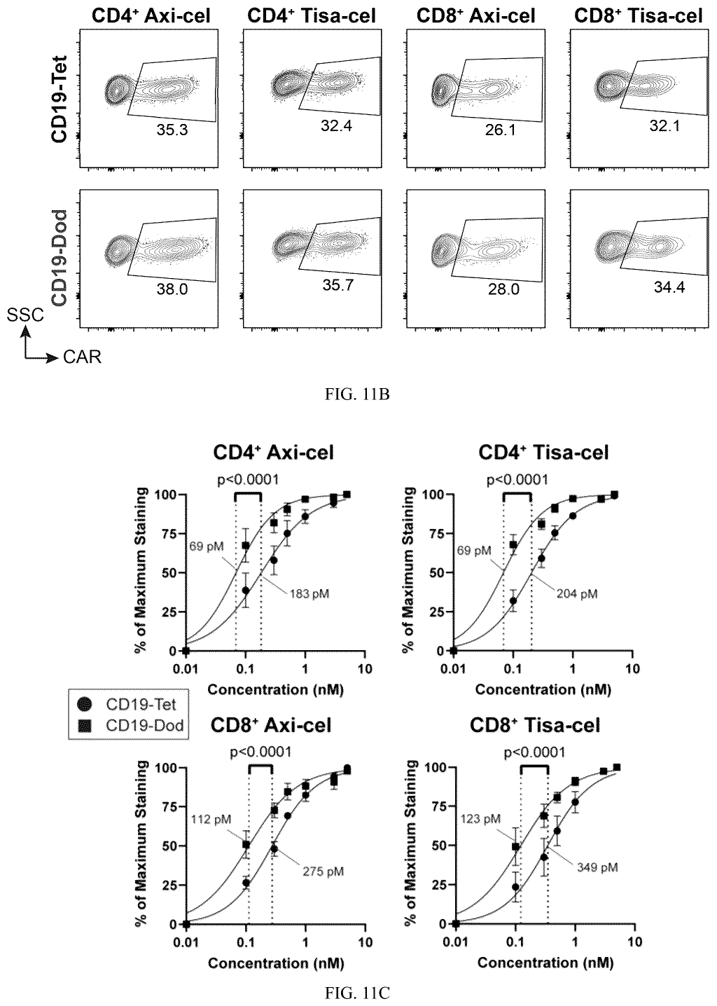

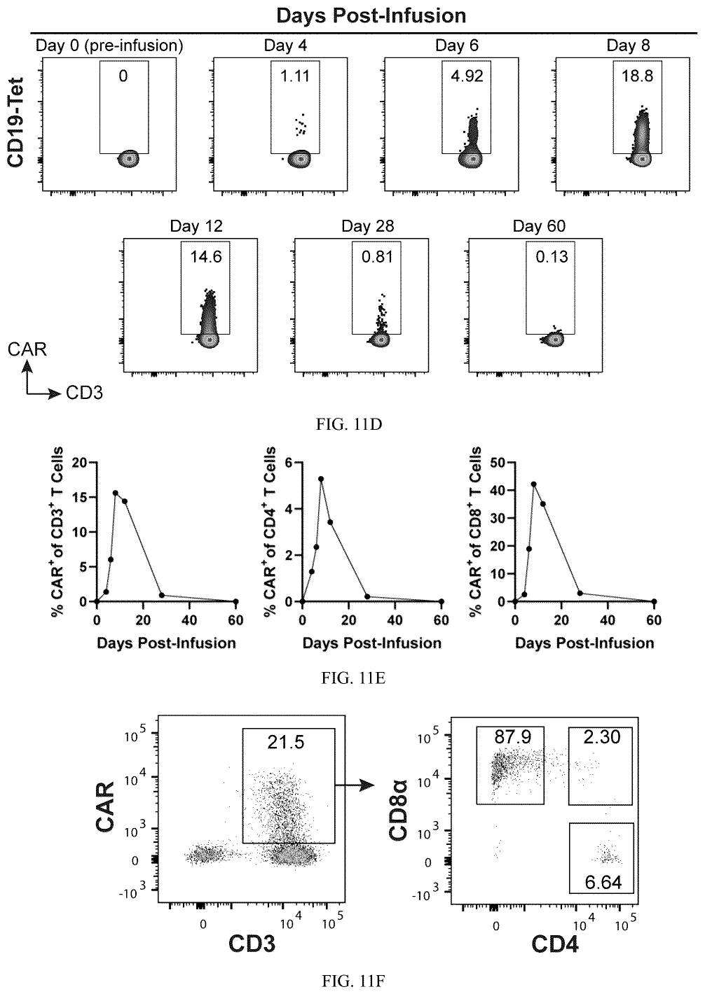

Following the validation of the applications of antigen-multimer in the CAR-transduced cell lines, the antigen-multimer is further applied to the clinical biopsy. The specimen is derived from the diffuse large B-cell lymphoma or B-cell acute lymphoblastic leukemia patients subject to anti CD19 CAR-T cell therapy. During the therapy, the CAR-T cells derived from cell infusion products arrive at the sites of tumor via peripheral circulation (FIG. 11A). The CD19 antigen-multimer is thus applied to the detection of CAR-T cells in the cell infusion product, peripheral blood, and tumor biopsy.

The CD19 antigen-multimer is titrated in the Axicabtagene Ciloleucel and Tisagenelecluel cell infusion product. At higher concentrations, the CD19 antigen-multimer can stain more CD4 and CD8 T cells from any one of the two infusion products. At the saturated concentrations, the CD19 antigen-streptavidin tetramer and CD19 antigen-dodecamer can detect the same percentiles of CAR: cells in 6 patients tested (FIG. 11B). The EC₅₀ value of staining is calculated by fitting the dose-effect curve with the relative staining percentiles. The staining EC₅₀ of tetramer is always higher than that of dodecamer (FIG. 11C), which is consistent with the observation in previously mentioned cell line study (FIG. 1D). In addition, the staining EC₅₀ of the two cell infusion products are comparable (FIG. 11C). Lastly, the staining EC₅₀ of CD8⁺ CAR-T cells is higher than that of CD4⁺ CAR-T cells. CD8⁺ CAR-T cells may express less CAR per cell, leading to lower detection with medium concentrations of CD19 antigen-multimer applied (FIG. 11C).

The peripheral blood is detected with CD19 antigen-tetramer at different time points post CAR-T cell infusion. The sample is from a patient subject to Axicabtagene Ciloleucel CAR-T cell infusion product treatment (FIG. 11D). The data show that the percentiles of CAR-T increase and achieve the peak at Day 9 post infusion. After that, the percentiles of CAR-T decrease, and are negligible at Day 60 post infusion (FIG. 11 D-E). Most of infused CAR-T cell express CD8. These observations are consistent with the predicted mode of CAR-T cell expansion and shrinkage. These findings demonstrates that CD19 antigen-streptavidin tetramer can precisely detect the CAR-T cells from patient specimen. Lastly, the tumor cell suspension from the patient subject to Axicabtagene Ciloleucel cell fusion product treatment for 14 days was detected with CD19 antigen-dodecamer (FIG. 11F). The CAR-T cells account for 22% of white cells, with 88% of CD8⁺ cells and 7% of CD4⁺ T cells, and small amount (2%) of CD4⁺CD8⁺ double-positive cells. These findings demonstrates that CD19 antigen-multimer can precisely detect the CD4⁺ and CD8⁺ CAR-T cells in the cell infusion product, peripheral blood and tumor biopsy of patients post infusion.

### Embodiment 8: Isolation of CAR-T Cells From Patient Specimen With Antigen-Multimer for Single-Cell Omics Analysis

Following the validation of applications of CD19 antigen-multimer in the detection of CAR-T cells from patient specimen, the CD19 antigen-streptavidin tetramer is then applied to isolate the anti CD19 CAR-T cells from the peripheral blood of patients post infusion to perform single cell omics analysis. The specimen is from one diffuse large B-cell lymphoma patient subject to Axicabtagene Ciloleucel CAR-T cell infusion product treatment. The patient achieves the clinical standard of complete response at Day 30 post infusion. The peripheral blood is collected at day 21 post infusion.

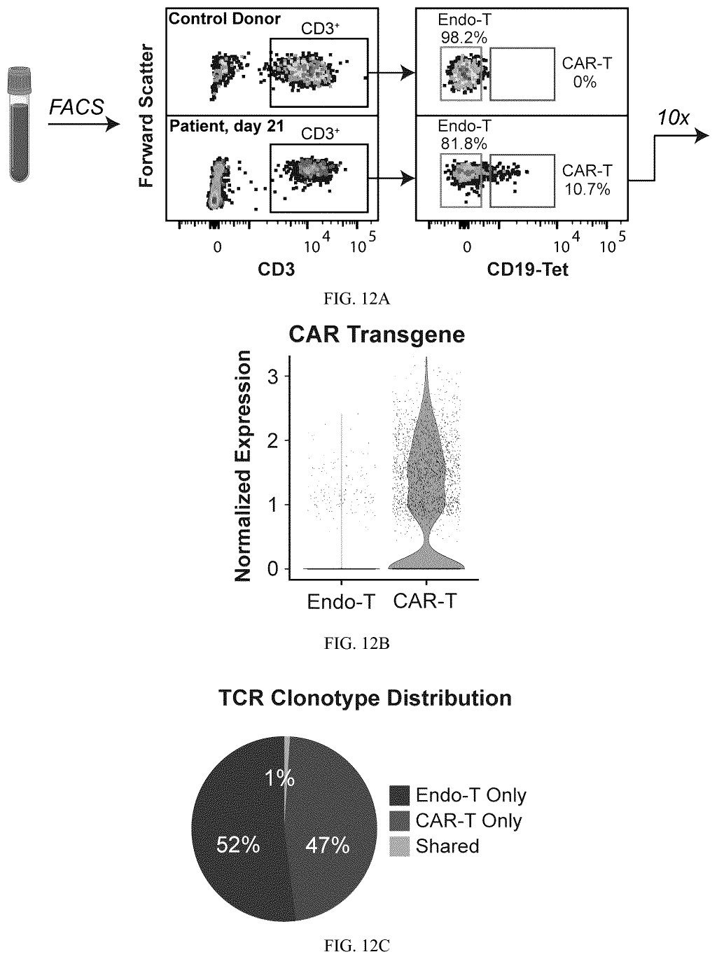

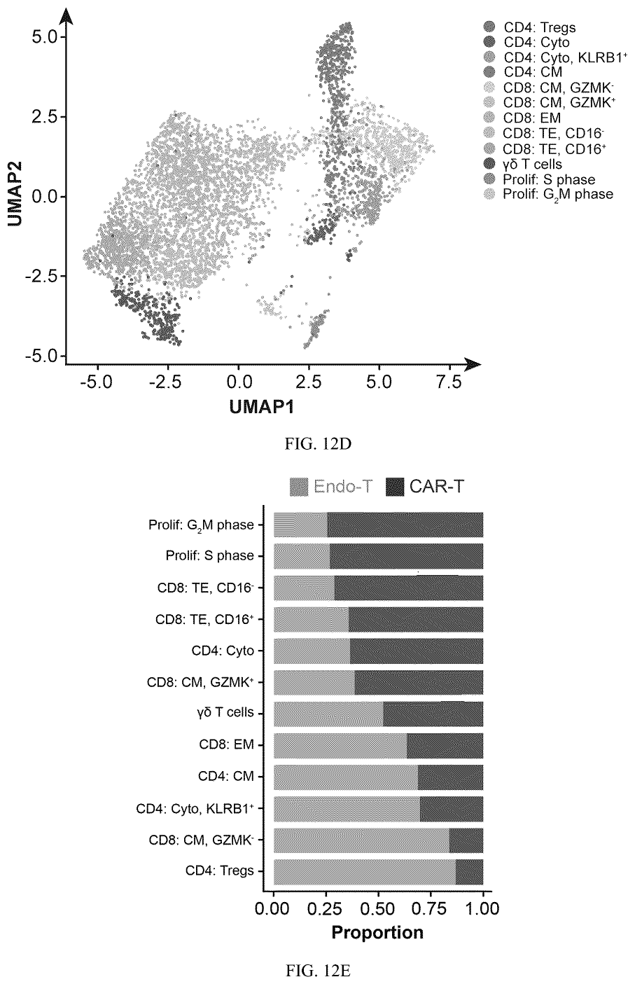

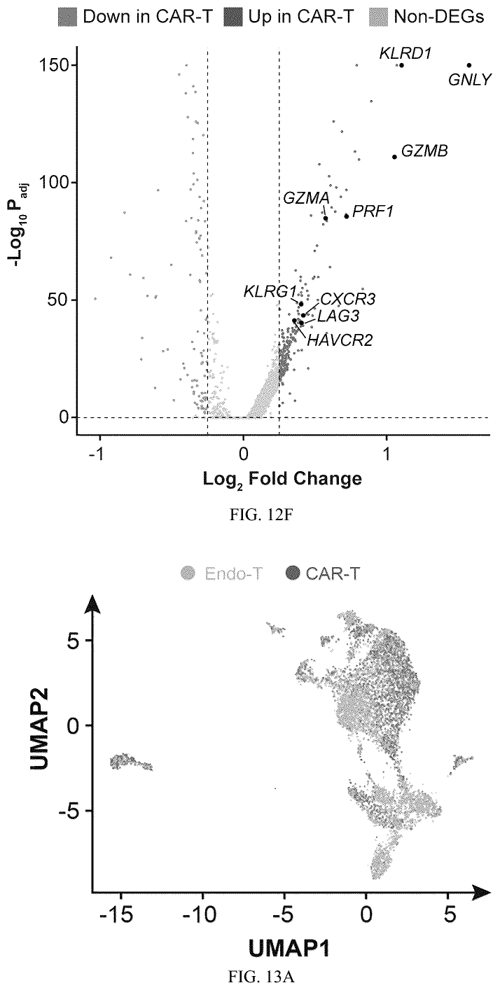

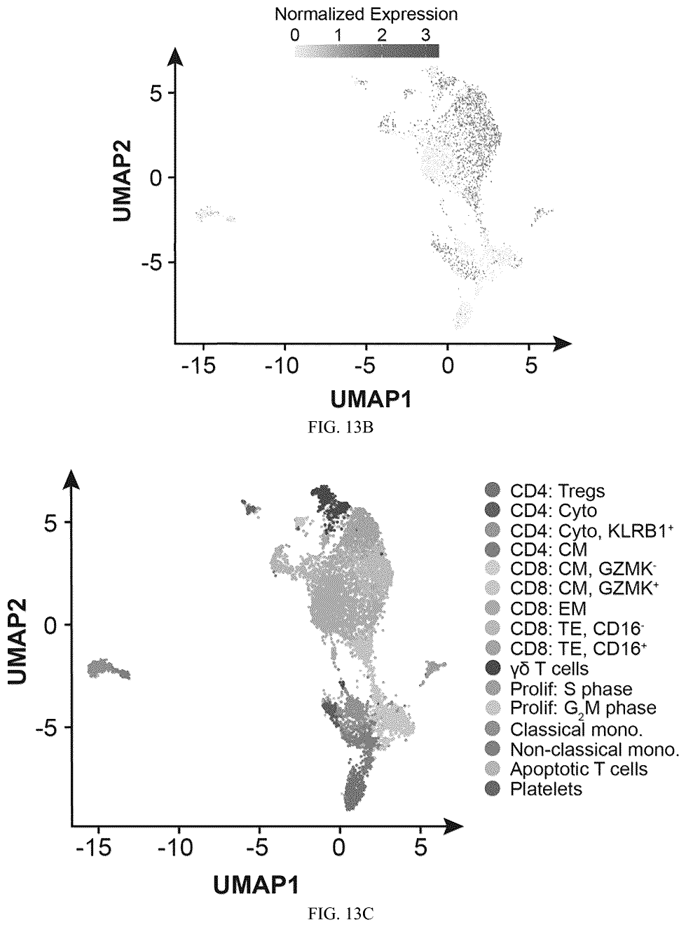

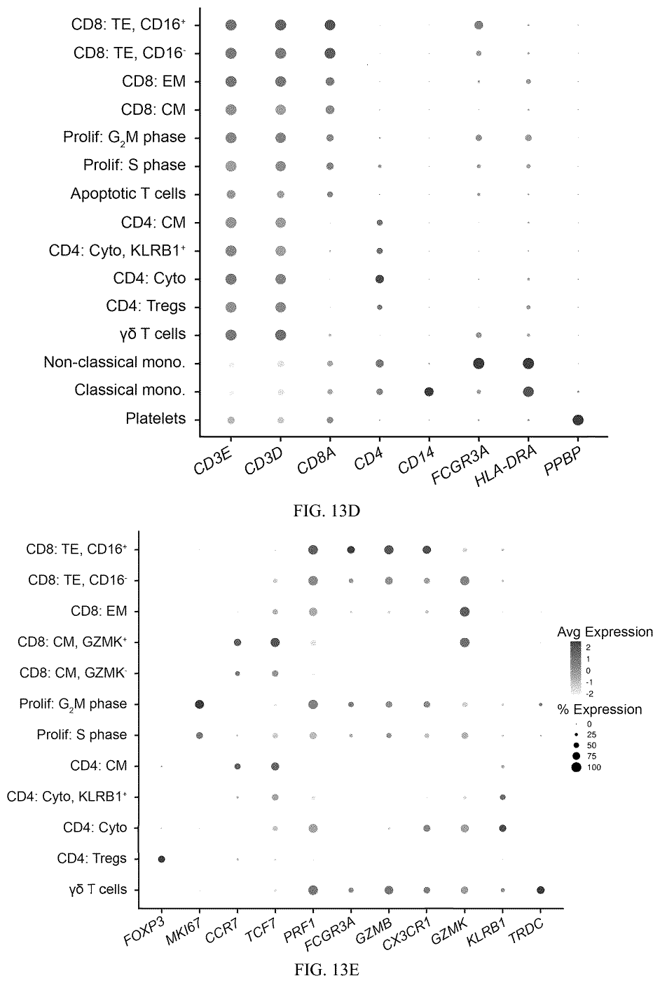

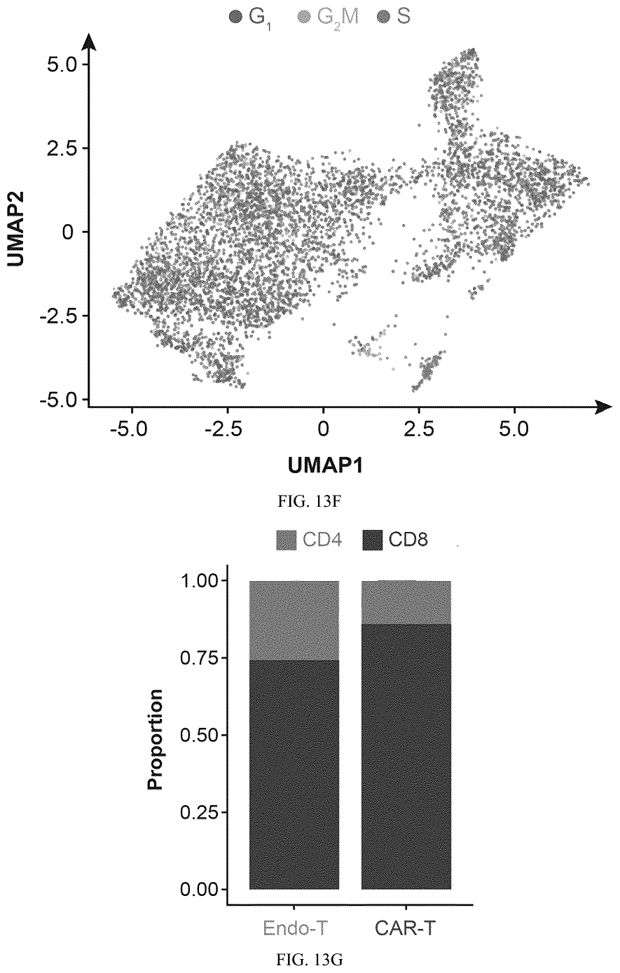

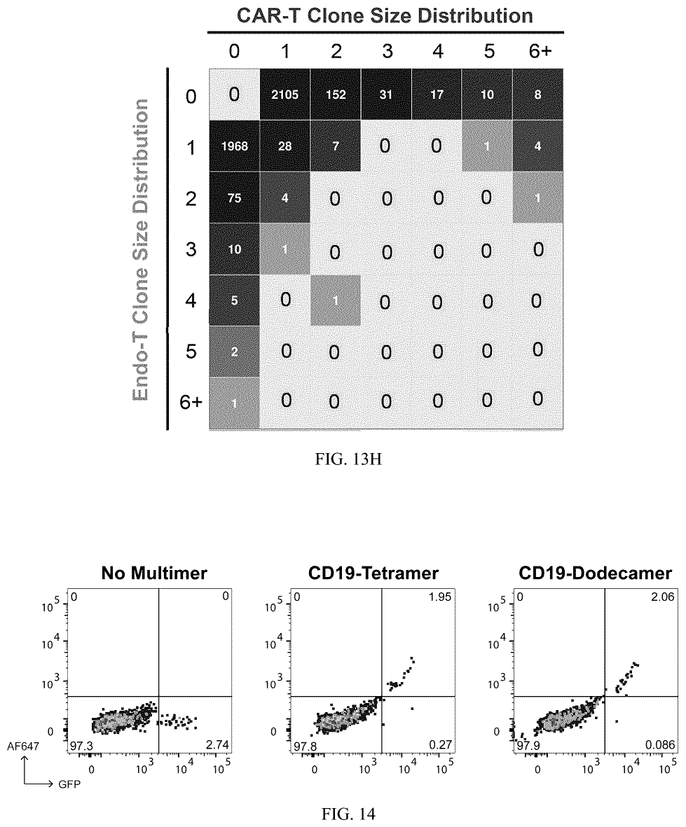

At Day 21 post infusion, the CD19 antigen-tetramer staining shows that 10.3% of CD3⁺ T cells are detected (CAR⁺ cells) (FIG. 12A). The CAR⁺ T cells (CAR-T) and endogenous CAR⁻ T cells (Endo-T) are sorted with flow cytometer and analyzed with matched single cell RNA and TCR sequencing. The single cell sequencing shows that the Axicabtagene Ciloleucel CAR gene transduction is highly CAR-T specific (FIG. 12B and FIG. 13A-B). This observation is in consistence with the properties of high specificity, sensitivity, and precision of CD19 antigen-streptavidin tetramer, and demonstrates that the antigen-multimer can be used to sort out the true CAR-T cell population with high purity. In addition, although various TCR clonotypes are found in CAR-T cells and Endo-T cells, the overlap of each other is limited and is ≤1% (FIG. 12C ad FIG. 13H). These findings demonstrates that CD19 antigen-streptavidin tetramer can capture distinct CAR-transduced T cell clones from patient specimen.

Using unified manifold approximation and projection (UMAP) and unsupervised Louvain clustering and filter-out of 3 non-T cell clusters, 12 T cell clusters are identified with known markers (including FOXP3, CCR7, TCF7, GZMB, KLRB1, and TRDC), which include proliferative, effect CD8⁺, cytotoxic CD4⁺, central memory, effect memory, γδ, and regulatory T cells (FIG. 12D and 13C-F). Compared to endogenous Endo-T cells, CAR-T cells are enriched in CD8⁺ T cells (FIG. 13G). in addition, CAR-T cells are enriched in proliferative and effect T cell clusters, while Endo-T cells are enriched in memory and regulatory T cell clusters (FIG. 12E). It is noteworthy that CAR-T cells are found both in γδ and regulatory T cells. The differential gene analysis shows that CAR-T cells have higher expression levels of activation genes (CXCR3, LAG3 and HAVCR2), cytotoxicity related genes (GNLY, KLRD1, GZMB, PRF1, and GZMA), and T cell aging-related genes (KLRG1) (FIG. 12F). These properties are in consistency with CAR-specific T cell activation, cytotoxicity, and differentiation. Taken together, antigen-multimer can be reliably applied to the isolation and analysis of CAR-T cells.

### Embodiment 9: CAR-NK Cells Are Detected With Antigen-Multimer

NK-92 cells are cultured with NK-92 medium (Alpha MEM, 12.5% FBS, 12.5% horse serum, 0.2 mM of i-inositol, 20 μM of folic acid, 2 mM of L-glutamine, 0.1 mM of β-mercaptothion) supplemented with 100 U/mL of human IL-2. 100,000 NK-92 cells are seeded in 12-well plate with 1 mL of NK-92 culture medium supplemented with 10 μg/mL of protamine sulfate and are transduced with CAR lentivirus at MOI 20. After three days of incubation, the CAR transduction efficiency is analyzed with flow cytometry.

The CAR-NK cells are stained with antigen-multimer (FIG. 14). 5,000 CAR-transduced NK92 cells are stained with 5 nM of CD19 antigen-streptavidin tetramer or CD19 antigen-dodecamer in FACS buffer (PBS, 2% FBS, 0.05% sodium azide) for 30 minutes, then stained with Near-IR Fixable Live/Dead Viability Dye in 1:1,000 dilutions in PBS for 5 minutes at 4° C. Finally, the cells are rinsed three times before analyzed with flow cytometry.

### Embodiment 10: Detection of CAR With Single-Cell Sequencing Total-Seq

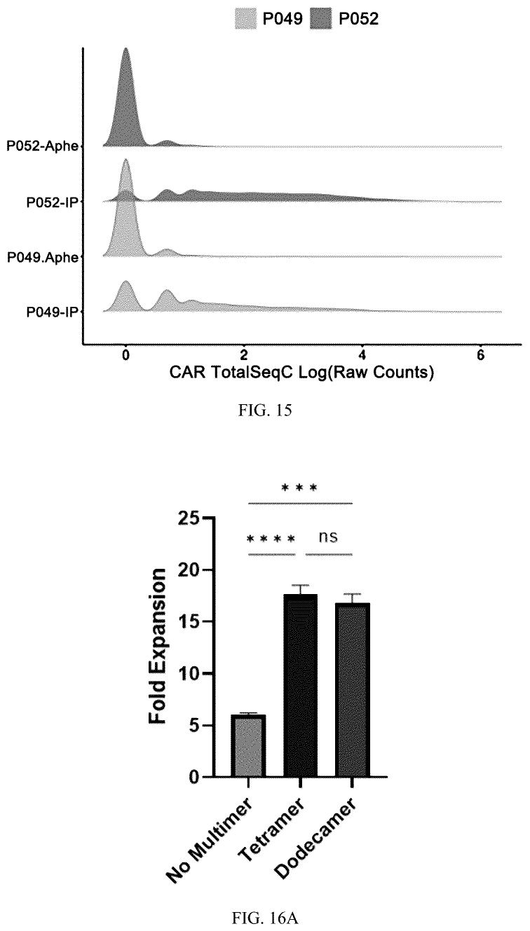

Diffuse large B cell lymphoma patient 049 (complete response) and 052 (no response) are subjected to Axicabtagene Ciloleucel anti-CD19 CAR-T cell therapy. The manufactured CAR-T cell infusion product is stained with 3 nM of oligo nucleotide-barcoded (Total-seq C, Biolegend, 405269) CD19 antigen-streptavidin tetramer. Then the cells are incubated with Near-IR Fixable Live/Dead Viability Dye at 1:1,000 dilutions in PBS for 5 minutes at 4° C. Finally, the cells are rinsed three times and resuspended in RMMI medium supplemented with 10% FBS. The single cell sequencing is performed using 10× Genomics 5′ kit. The data is analyzed with Cell Ranger. The results show that the antigen-multimer can be applied in CAR detection with single cell sequencing method of Total-seq (FIG. 15).

### Embodiment 11: The Antigen-Multimer Promotes the CAR-T Cell Expansion and Activation

Experimental protocol: The healthy donor-derived T cells are transduced with second-generation CD19 CAR (clone FMC63) at MOI 5. The CAR-transduced cells are cultured in T cell culture medium (RPMI medium, supplemented with 10% FBS, 2 mM of L-glutamine, 50 uM of 2-methanol) and 50 U/mL of IL-2 for 12 days. The transfection efficiency of CAR is about 50%. 2×10⁴ CAR⁺ T cells are incubated under the following conditions: 1) without multimer; 2) 5 nM of CD19 antigen-streptavidin tetramer; 3) 5 nM of CD19 antigen-dodecamer. After culture with 50 U/mL of IL-2 for 6 days, the following markers and fluorescence are analyzed with flow cytometry: CAR (meGFP), CD3 (BV421), CD4 (BV510), CD8α (PCP/Cy5.5), CD69 (PE), CD25 (AF647), PD-1 (BV711), and Near-IR Live/Dead Cell Staining.

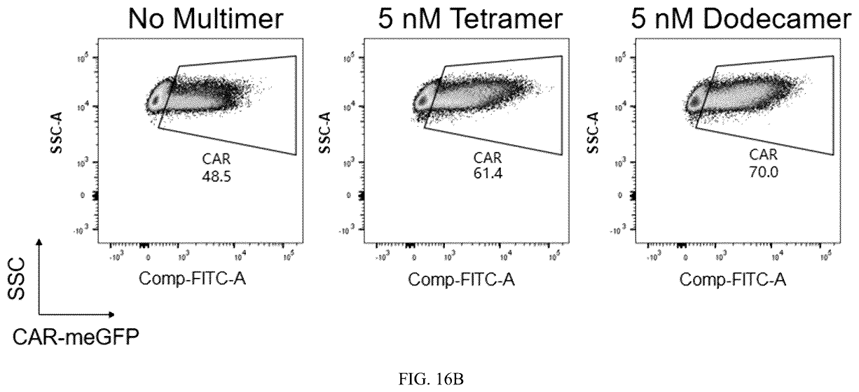

Experimental results: For cell expansion, both tetramer and dodecamer treatment can significantly expand the number of CAR-T cells at 15-20 folds, while without multimer supplementation the number of CAR-T cells only increase by about 5 folds (p<0.001) (FIG. 16A). No significance is observed between the two groups of tetramer and dodecamer treatment. In addition, CAR-T cells are enriched under multimer treatment. CAR-T cells account for 49% of total cell population when multimer is not supplemented, increase to 61% under tetramer treatment, and increase to 70% under dodecamer treatment (FIG. 16B).

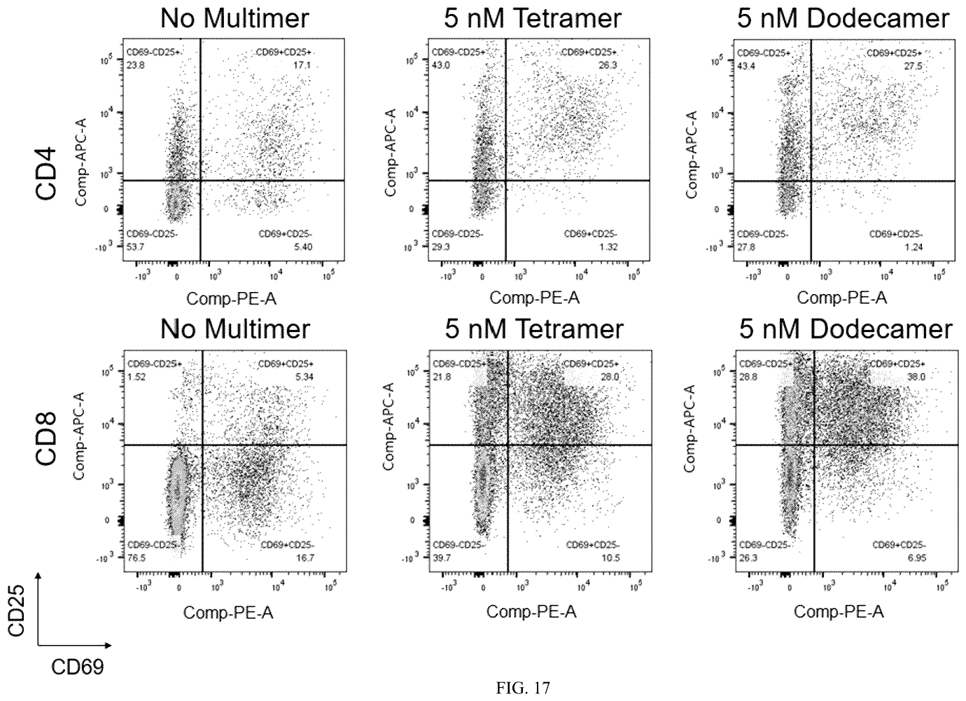

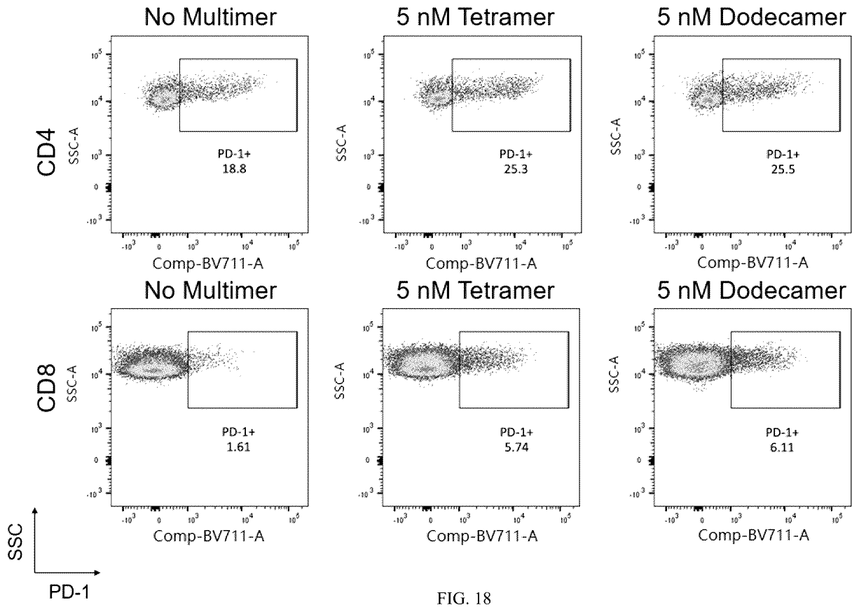

For the outcome of activation, both tetramer and dodecamer up regulate CD69 (early activation marker) and CD25 (late activation marker) expression (FIG. 17). In CD4⁺ CAR-T cell sub-population, CD69⁺CD25⁺ cells account for 17% of total cell population when multimer is not supplemented, increase to 26% under tetramer treatment, and increase to 28% under dodecamer treatment. In CD8⁺ CAR-T cell sub-population, CD69⁺CD25⁺ cells account for 5% of total cell population when multimer is not supplemented, increase to 28% under tetramer treatment, and increase to 38% under dodecamer treatment. In addition, PD-1 expression is up regulated under multimer treatment (FIG. 18). In CD4⁺ CAR-T cell sub-population, PD-1⁺ CAR-T cells account for 25% of total cell population when multimer is not supplemented, increase to 26% under tetramer treatment, and increase to 25% under dodecamer treatment. In CD8⁺ CAR-T cell sub-population, PD-1⁺ CAR-T cells account for 2% of total cell population when multimer is not supplemented, increase to 6% under tetramer treatment, and increase to 6% under dodecamer treatment.

Conclusions: Both tetramer and dodecamer treatment can selectively expand CAR-T cells. After six days of treatment, the cell numbers and percentiles of CAR-T cells both significantly increase. The treatment of tetramer or dodecamer up regulate the activation markers (CD69, CD25 and PD-1). It is more significant for CD8+ cell population to be activated. For the markers of CD69 and CD25, the effect of dodecamer treatment is more significant, compared to tetramer treatment.

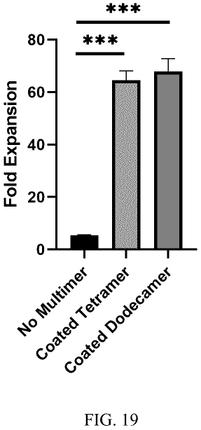

Furthermore, the CAR-T cell expansion is promoted by coating the multimers onto the cell culture surface. Both the coated tetramer and dodecamer can significantly expand the number of CAR-T cells at 60-70 folds, while without multimer the number of CAR-T cells only increase by about 5 folds (p<0.001) (FIG. 19).

The results demonstrates that antigen-multimer can be used to expand and activate CAR-T cells. This property is very important in the manufacturing of CAR-T cells. The observed superior effect of dodecamer in activation of CD8+CAR-T cells than tetramer, may be caused by its higher binding avidity.

What is claimed is:

1. A method for labeling chimeric antigen receptor, the method uses a reagent to label a chimeric antigen receptor to be detected; the reagent comprises a complex of antigens and a multimer;
the antigen is a biotin-tagged antigen, which can specially bind to the chimeric antigen receptor;
the multimer, comprising:
a scaffold protein, which is comprised of four protein subunits; a scaffold protein is composed of functional protein subunits; or a scaffold protein is composed of a mixture of functional and dysfunctional protein subunits; the functional protein subunit is derived from a streptavidin and has no biotin-binding activity; the scaffold protein has at least one functional protein subunit; the functional protein subunit's C-terminus has a cysteine and a biotin tag; the amino acid sequence of the functional protein subunit is set forth in SEQ ID NO: 1 or SEQ ID NO:2; the number of the dysfunctional protein subunit is not greater than 2, and the amino acid sequence is set forth in SEQ ID NO:3;
a streptavidin, binding to the biotin tag of the scaffold protein, wherein the streptavidin is used to bind 1˜3 of the biotin-tagged antigens; the streptavidin has a detectable label or detectable labels.

2. The method of claim 1, wherein the affinity KD value of the chimeric antigen receptor to be detected is not less than 0.1 nM.

3. The method of claim 1, wherein the detectable label is selected from: fluorochromes, nucleic acids, proteins, lipids, glycans, enzymes, fats, membranes, polysaccharides, metals, radioisotopes, nucleotides, peptides, sugars, fatty acids, amino acids, inorganic molecules, and organic molecules.

4. A method for specific expansion of chimeric antigen receptor expressing cells, the method comprises the following steps: supplementing an expansion reagent to a cultured CAR-T cell population; and rendering the cultured CAR-T cell population to expand to meet a preset standard of therapy;
wherein the expansion reagent comprises a complex of antigens and a multimer;
the antigen, is expressed on a target cell surface, and is specifically recognized by a chimeric antigen receptor;
the multimer, comprising:
a scaffold protein, which is comprised of four protein subunits; a scaffold protein is composed of functional protein subunits; or a scaffold protein is composed of a mixture of functional and dysfunctional protein subunits; the functional protein subunit is derived from a streptavidin and has no biotin-binding activity; the scaffold protein has at least one functional protein subunit; the functional protein subunit's C-terminus has a cysteine and a biotin tag; the amino acid sequence of the functional protein subunit is set forth in SEQ ID NO: 1 or SEQ ID NO:2; the number of the dysfunctional protein subunit is not greater than 2, and the amino acid sequence is set forth in SEQ ID NO: 3;
a streptavidin, binding to the biotin tag of the scaffold protein, wherein the streptavidin is used to bind 1˜3 of the antigens via biotin;
or the expansion reagent comprises a streptavidin; the streptavidin binds to 2 to 4 antigens via biotin; the antigen, is expressed on a target cell surface and is specifically recognized by a chimeric antigen receptor.

5. A method for detection of chimeric antigen receptor expression, the method comprises the following steps: using a detection reagent to stain a chimeric antigen receptor, and an expression of the chimeric antigen receptor is measured with single-cell sequencing;
wherein the detection reagent is an antigen-multimer which is labeled with oligo nucleotide, the antigen-multimer comprises a complex of antigens and a multimer;
the antigen, is expressed on a target cell surface, and is specifically recognized by a chimeric antigen receptor;
the multimer, comprising:
a scaffold protein, which is comprised of four protein subunits; a scaffold protein is composed of functional protein subunits; or a scaffold protein is composed of a mixture of functional and dysfunctional protein subunits; the functional protein subunit is derived from a streptavidin and has no biotin-binding activity; the scaffold protein has at least one functional protein subunit, the functional protein subunit's C-terminus has a cysteine and a biotin tag; the amino acid sequence of the functional protein subunit is set forth in SEQ ID NO:1 or SEQ ID NO:2; the number of the dysfunctional protein subunit is not greater than 2, and the amino acid sequence is set forth in SEQ ID NO:3;
a streptavidin, binding to the biotin tag of the scaffold protein, wherein the streptavidin is used to bind 1˜3 of the antigens via biotin; the streptavidin has detectable labels;
or the detection reagent comprises a streptavidin; the streptavidin is unlabeled, fluorescence-labeled, or oligo nucleotide-labeled; the streptavidin binds to 2 to 4 antigens via biotin; the antigen, is expressed at a surface of a target cell, and is specifically recognized by a chimeric antigen receptor.


SEQUENCE LISTING (7 sequences)

SEQ ID NO: 1 - protein, 128 aa, synthetic construct
AEAGITGTWYAQLGDTFIVTAGADGALTGTYEAAVGNAESRYVLTGRYDSAPATDGSGTALGWTVAWKNNYRNAHSATTWSGQYVGGAEARINTQWLLTSGTTEANAWKSTLVGHDTFTKVKPSAASC

SEQ ID NO: 2 - protein, 133 aa, synthetic construct
AEAGITGTWYAQLGDTFIVTAGADGALTGTYEAAVGNAESRYVLTGRYDSAPATDGSGTALGWTVAWKNNYRNAHSATTWSGQYVGGAEARINTQWLLTSGTTEANAWKSTLVGHDTFTKVKPSAASGGGGSC

SEQ ID NO: 3 - protein, 133 aa, synthetic construct
AEAGITGTWYAQLGDTFIVTAGADGALTGTYEAAVGNAESRYVLTGRYDSAPATDGSGTALGWTVAWKNNYRNAHSATTWSGQYVGGAEARINTQWLLTSGTTEANAWKSTLVGHDTFTKVKPSAASHHHHHH

SEQ ID NO: 4 - protein, 321 aa, synthetic construct
METDTLLLWVLLLWVPGSTGDVDEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKQRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARGSGLNDIFEAQKIEWHEKLGLEVLFQGPELEHHHHHHHHHH

SEQ ID NO: 5 - protein, 693 aa, synthetic construct
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTGSGLNDIFEAQKIEWHEKLGLEVLFQGPELEHHHHHHHHHH

SEQ ID NO: 6 - protein, 8 aa, synthetic construct
CARBOHYD

SEQ ID NO: 7 - protein, 39 aa, synthetic construct
GLNDIFEAQKIEWHEKLGLEVLFQGPELEHHHHHHHHHH